(19) United States
(12) Patent Application Publication
(10) Pub. No.: US 2004/0224972 A1
(43) Pub. Date: Nov. 11, 2004

(54) Medicinal compositions for concomitant use as anticancer agent

(76) Inventors: Yoichi Ozawa, Ibaraki (JP); Kentaro Yshimatsu, Ibaraki (JP); Junko Kai, Ibaraki (JP)

Correspondence Address: BIRCH STEWART KOLASCH & BIRCH, PO BOX 747, FALLS CHURCH, VA, 22040-0747, US

(21) Appl. No.: 10/839,222

(22) Filed: May 6, 2004

Related U.S. Application Data
(63) Continuation of application No. PCT/JP01/09563, filed on Oct. 31, 2001.

(30) Foreign Application Priority Data
Oct. 31, 2000 (JP) 2000-333952

(51) Int. Cl. A61K 31/4745; A61K 31/18

(52) U.S. Cl. 514/300; 514/419; 514/602

(57) ABSTRACT

The present invention provides a medicinal composition having an excellent antitumor activity. That is, it provides a medicinal composition comprising a sulfonamide compound, a sulfonate compound or a salt of them, which is represented by the following formula:

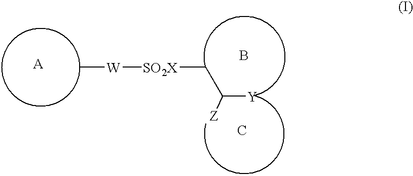

(wherein ring A represents an aromatic ring which may have a substituent group; ring B represents a 6-membered unsaturated hydrocarbon ring which may have a substituent group etc.; ring C represents a 5-membered hetero-ring containing one or two nitrogen atoms, and the ring C may have a substituent group; W represents a single bond or —CH═CH—; X represents —NH— etc.; and Y represents a carbon atom or a nitrogen atom; and Z represents —NH— etc.), particularly N-(3-chloro-1H-indol-7-yl)-4-sulfamoylbenzenesulfonamide or a salt thereof, combined with at least one substance selected from (1) irinotecan hydrochloride trihydrate; (2) mitomycin C; (3) 5-fluorouracil; (4) cisplatin; (5) gemcitabine hydrochloride; (6) doxorubicin; (7) taxol; (8) carboplatin; (9) oxaliplatin; (10) capecitabine; and (11) a salt of the above-mentioned (1) to (10).

### TECHNICAL FIELD

The present invention relates to a novel medicinal composition comprising a compound useful as an anticancer drug, particularly N-(3-chloro-1H-indol-7-yl)-4-sulfamoylbenzenesulfonamide or a pharmacologically acceptable salt thereof, combined with another anticancer agent.

### PRIOR ART

As chemicals used conventionally as chemotherapeutic drugs for cancers, there are a large number of chemicals such as cyclophosphamide as an alkylating agent, methotrexate and fluorouracil as antimetabolites, doxorubicin, mitomycin and bleomycin as antibiotics, vincristine and etoposide derived from plant, and cisplatin as a metal complex, but their antitumor effects are insufficient, so there is a demand for development of new antitumor agents.

On one hand, the present inventors succeeded for the first time in synthesis of completely new aromatic sulfonamides or aromatic sulfonate compounds, they unexpectedly found that these compounds exhibit an excellent antitumor activity, and they provided useful antitumor agents showing new mechanism of action (JP-A 7-165708). In particular, N-(3-chloro-1H-indol-7-yl)-4-sulfamoylbenzenesulfonamide (hereinafter, also called “E7070”) represented by the following

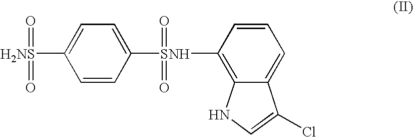

exhibits an activity on various types of tumors and is very useful.

There is the case where a certain kind of compound is effective against a certain kind of cancer but poor in effectiveness against other cancers. Further, administration of a single component may not achieve a sufficient effect. That is, the object of the present invention is to provide a medicinal composition having an excellent antitumor activity capable of solving these problems.

### Disclosure of the Invention

In view of the foregoing, the present inventors made extensive study to search for an excellent antitumor composition, and as a result, they unexpectedly found that a synergistically further excellent antitumor activity can be achieved by using a certain anticancer agent in combination with a sulfonamide compound, a sulfonate compound or a salt of them (sulfone compound (I)), which is represented by the following formula:

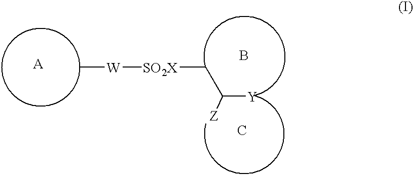

(wherein ring A represents a monocyclic aromatic ring or a bicyclic aromatic ring, each of which may have a substituent group; ring B represents a 6-membered unsaturated hydrocarbon ring or a 6-membered unsaturated hetero ring containing one nitrogen atom, each of which may have a substituent group; ring C represents a 5-membered hetero ring containing one or two nitrogen atoms, and the ring C may have a substituent group; W represents a single bond or —CH═CH—; X represents —N(R¹)—(wherein R¹ represents a hydrogen atom or a lower alkyl group) or an oxygen atom; Y represents a carbon atom or a nitrogen atom; Z represents —N(R₂)— (wherein R₂ represents a hydrogen atom or a lower alkyl group) or a nitrogen atom, provided that the above-defined compounds exclude compounds in the following cases (a) and (b):

(a) ring A is 4-methylbenzene; W is a single bond; X is —NH—; ring B is methoxy benzene; and ring C is unsubstituted imidazole; and (b) ring A is 4-(acetamide)benzene or 4-aminobenzene; W is a single bond; X is —NH—; ring B is unsubstituted benzene, and ring C is unsubstituted pyrazole), particularly with N-(3-chloro-1H-indol-7-yl)-4-sulfamoylbenzenesulfonamide or a salt thereof, and the present invention was thereby completed.

That is, the present invention relates to:

(1) a medicinal composition comprising the above-mentioned compound (I), combined with and at least one substance (substance (III)) selected from (a) irinotecan hydrochloride trihydrate; (b) mitomycin C; (c) 5-fluorouracil; (d) cisplatin; (e) gemcitabine hydrochloride; (f) doxorubicin; (g) taxol; and (h) a salt of the above-mentioned (a) to (g); (2) the medicinal composition described in the above (1), wherein in the above formula (I), ring A is benzene or pyridine, each of which may have a substituent group; ring B is benzene which may have a substituent group, ring C ispyrrole which may have a substituent group; W is a single bond; and each of X and Z is —NH—; (3) the medicinal composition described in the above (1), which is an anticancer agent; (4) the medicinal composition described in the above (3), which comprises N-(3-chloro-1H-indol-7-yl)-4-sulfamoylbenzenesulfonamide (E7070) or a salt thereof, and at least one substance selected from (a) irinotecan hydrochloride trihydrate; (b) mitomycinC; (c) 5-fluorouracil; (d) cisplatin; (e) gemcitabine hydrochloride; (f) doxorubicin; (g) taxol; and (h) a salt of the above-mentioned (a) to (g); (5) the medicinal composition described in the above (3), which comprises E7070 or a salt thereof, and irinotecan hydrochloride trihydrate or a salt thereof; (6) the medicinal composition described in the above (3), which comprises E7070 or a salt thereof, and mitomycin C or a salt thereof; (7) the medicinal composition described in the above (3), which comprises E7070 or a salt thereof, and 5-fluorouracil or a salt thereof; (8) the medicinal composition described in the above (3), which comprises E7070 or a salt thereof, and cisplatin or a salt thereof; (9) the medicinal composition described in the above (3), which comprises E7070 or a salt thereof, and gemcitabine hydrochloride or a salt thereof; (10) the medicinal composition described in the above (3), which comprises E7070 or a salt thereof, and doxorubicin or a salt thereof; (11) the medicinal composition described in the above (3), which comprises E7070 or a salt thereof, and taxol or a salt thereof; (12) the medicinal composition described in the above (3), which comprises E7070 or a salt thereof, irinotecan hydrochloride trihydrate or a salt thereof, and cisplatin or a salt thereof; (13) the medicinal composition described in the above (3), which comprises E7070 or a salt thereof, irinotecan hydrochloride trihydrate or a salt thereof, 5-fluorouracil or a salt thereof, and cisplatin or a salt thereof; (14) use of E7070 or a salt thereof for producing a medicinal composition comprising it in combination with at least one substance selected from (a) irinotecan hydrochloride trihydrate; (b) mitomycin C; (c) 5-fluorouracil; (d) cisplatin; (e) gemcitabine hydrochloride; (f) doxorubicin; (g) taxol; and (h) a salt of the above-mentioned (a) to (g); (15) use of at least one substance selected from (a) irinotecan hydrochloride trihydrate; (b) mitomycin C; (c) 5-fluorouracil; (d) cisplatin; (e) gemcitabine hydrochloride; (f) doxorubicin; (g) taxol; and (h) a salt of the above-mentioned (a) to (g), for producing a medicinal composition comprising it in combination with E7070 or a salt thereof; (16) use of E7070 or a salt thereof, and at least one substance selected from (a) irinotecan hydrochloride trihydrate; (b) mitomycin C; (c) 5-fluorouracil; (d) cisplatin; (e) gemcitabine hydrochloride; (f) doxorubicin; (g) taxol; and (h) a salt of the above-mentioned (a) to (g), for producing an agent for preventing or treating a cancer; (17) a method for preventing or treating a cancer, by administrating an effective amount of the medicinal composition described in the above (3) to a patient; (18) a method for preventing or treating a cancer, by administering E7070 or a salt thereof to a patient, and then administering, after a predetermined time, at least one substance selected from (a) irinotecan hydrochloride trihydrate; (b) mitomycin C; (c) 5-fluorouracil; (d) cisplatin; (e) gemcitabine hydrochloride; (f) doxorubicin; (g) taxol; and (h) a salt of the above-mentioned (a) to (g) to the patient; (19) a method for preventing or treating a cancer, by administering at least one substance selected from (a) irinotecan hydrochloride trihydrate; (b) mitomycin C; (c) 5-fluorouracil; (d) cisplatin; (e) gemcitabine hydrochloride; (f) doxorubicin; (g) taxol; and (h) a salt of the above-mentioned (a) to (g) to a patient, and then administering, after a predetermined time, E7070 or a salt thereof to the patient; (20) a pharmaceutical kit for administering effective amounts of the above-mentioned sulfone compound (I) and the above-mentioned substance (III) simultaneously or separately to a patient; (21) a pharmaceutical product comprising the above-mentioned sulfone compound (I) combined with the above-mentioned substance (III); (22) a combination comprising the above-mentioned sulfone compound (I) combined with the above-mentioned substance (III); (23) a method for preventing or treating a cancer, by administering effective amounts of the above-mentioned sulfone compound (I) and the above-mentioned substance (III) simultaneously or separately to a patient; and (24) a medicinal composition comprising effective amounts of the above-mentioned sulfone compound (I) and the above-mentioned substance (III).

In addition, the invention provides (25) a medicinal composition comprising the above-mentioned compound (1), combined with and at least one substance (substance (IV)) selected from (a) irinotecan hydrochloride trihydrate; (b) mitomycinC; (c) 5-fluorouracil; (d) cisplatin; (e) gemcitabine hydrochloride; (f) doxorubicin; (g) taxol; (h) carboplatin; (i) oxaliplatin; (j) capecitabine; and (k) a salt of the above-mentioned (a) to (j); (26) the medicinal composition described in the above (25), wherein in the above formula (I), ring A is benzene or pyridine, each of which may have a substituent group; ring B is benzene which may have a substituent group, ring C is pyrrole which may have a substituent group; W is a single bond; and each of X and Z is —NH—; (27) the medicinal composition described in the above (25), which is an anticancer agent; (28) the medicinal composition described in the above (27), which comprises N-(3-chloro-1H-indol-7-yl)-4-sulfamoylbenzenesulfonamide (E7070) or a salt thereof, and at least one substance selected from (a) irinotecan hydrochloride trihydrate; (b) mitomycinC; (c) 5-fluorouracil; (d) cisplatin; (e) gemcitabine hydrochloride; (f) doxorubicin; (g) taxol; (h) carboplatin; (i) oxaliplatin; (j) capecitabine; and (k) a salt of the above-mentioned (a) to (j); (29) the medicinal composition described in the above (27), which comprises E7070 or a salt thereof, and carboplatin or a salt thereof; (30) the medicinal composition described in the above (27), which comprises E7070 or a salt thereof, and oxaliplatin or a salt thereof; (31) the medicinal composition described in the above (27), which comprises E7070 or a salt thereof, and capecitabine or a salt thereof; (32) the medicinal composition described in the above (27), which comprises E7070 or a salt thereof, 5-fluorouracil or a salt thereof, and oxaliplatin or a salt thereof; (33) use of E7070 or a salt thereof for producing a medicinal composition comprising it in combination with at least one substance selected from (a) irinotecan hydrochloride trihydrate; (b) mitomycin C; (c) 5-fluorouracil; (d) cisplatin; (e) gemcitabine hydrochloride; (f) doxorubicin; (g) taxol; (h) carboplatin; (i) oxaliplatin; (j) capecitabine; and (k) a salt of the above-mentioned (a) to (j); (34) use of at least one substance selected from (a) irinotecan hydrochloride trihydrate; (b) mitomycinC; (c) 5-fluorouracil; (d) cisplatin; (e) gemcitabine hydrochloride; (f) doxorubicin; (g) taxol; (h) carboplatin; (i) oxaliplatin; (j) capecitabine; and (k) a salt of the above-mentioned (a) to (j), for producing a medicinal composition comprising it in combination with E7070 or a salt thereof; (35) use of E7070 or a salt thereof, andat least one substance selected from (a) irinotecan hydrochloride trihydrate; (b) mitomycin C; (c) 5-fluorouracil; (d) cisplatin; (e) gemcitabine hydrochloride; (f) doxorubicin; (g) taxol; (h) carboplatin; (i) oxaliplatin; (j) capecitabine; and (k) a salt of the above-mentioned (a) to (j), for producing an agent for preventing or treating a cancer; (36) a method for preventing or treating a cancer, by administrating an effective amount of the medicinal composition described in the above (27) to a patient; (37) a method for preventing or treating a cancer, by administering E7070 or a salt thereof to a patient, and then administering, after a predetermined time, at least one substance selected from (a) irinotecan hydrochloride trihydrate; (b) mitomycin C; (c) 5-fluorouracil; (d) cisplatin; (e) gemcitabine hydrochloride; (f) doxorubicin; (g) taxol; (h) carboplatin; (i) oxaliplatin; (j) capecitabine; and (k) a salt of the above-mentioned (a) to (j) to the patient; (38) a method for preventing or treating a cancer, by administering at least one substance selected from (a) irinotecan hydrochloride trihydrate; (b) mitomycin C; (c) 5-fluorouracil; (d) cisplatin; (e) gemcitabine hydrochloride; (f) doxorubicin; (g) taxol; (h) carboplatin; (i) oxaliplatin; (j) capecitabine; and (k) a salt of the above-mentioned (a) to (j) to a patient, and then administering, after a predetermined time, E7070 or a salt thereof to the patient; (39) a pharmaceutical kit for administering effective amounts of the above-mentioned sulfone compound (I) and the above-mentioned substance (IV) simultaneously or separately to a patient; (40) a pharmaceutical product comprising the above-mentioned sulfone compound (I) combined with the above-mentioned substance (IV); (41) a combination comprising the above-mentioned sulfone compound (I) combined with the above-mentioned substance (IV); (42) a method for preventing or treating a cancer, by administering effective amounts of the above-mentioned sulfone compound (I) and the above-mentioned substance (IV) simultaneously or separately to a patient; and (43) a medicinal composition comprising effective amounts of the above-mentioned sulfone compound (I) and the above-mentioned substance (IV).

The medicinal composition, the use of the compound for producing the medicinal composition, the method for preventing or treating, the pharmaceutical kit, the pharmaceutical product, and the combination according to the present invention can be practiced respectively in methods described later.

Hereinafter, the meanings of symbols, terms etc. used in this specification are described, and the present invention is described in detail.

In the specification of this application, although structural formula of a compound may express a certain isomer for the sake of convenience, it goes without saying that the compounds contained in the medicinal composition according to the present invention are not limited to those of the structural formulae shown for convenience's sake and all possible isomers which can occur in the structures, for example geometric isomer, optical isomer based on asymmetrical carbon, rotational isomer, stereoisomer and tautomer, as well as a mixture of such isomers are included. Further, as the above-mentioned compound, crystal polymorphism may be present but, again, there is no limitation but any of single crystal form or a mixture will do. Further, the above-mentioned compound may be an anhydride or a hydrate, and either of them are included in the scope of claim for patent in the present invention. The medicinal composition according to the present invention exhibits a strong antitumor activity, and it also includes derivatives of the above-mentioned, which exhibit an antitumor activity as a result of metabolism such as oxidation, reduction, hydrolysis and conjugation in vivo. Further, the present invention also includes the compounds which produce the compound in the composition of the present invention as a result of metabolism such as oxidation, reduction and hydrolysis in vivo.

The “monocyclic aromatic ring or bicyclic aromatic ring” “used in this specification refers to a monocyclic or bicyclic aromatic hydrocarbon ring containing 6 to 14 carbon atoms or an aromatic hetero ring containing one or more hetero atoms selected from N, O and S. Preferable examples of the group include benzene, indene group, 1-naphthalene, 2-naphthalene, azulene, heptalene, cyclopentacyclooctene, benzocyclooctene, pyrrole, pyridine, pyridazine, pyrimidine, pyrazine, triazole, tetrazole, benzotriazole, pyrazole, imidazole, benzimidazole, indole, isoindole, indolizine, purine, indazole, quinoline, isoquinoline, quinolizine, phthalazine, naphthylidine, quinoxaline, quinazoline, cynnoline, pteridine, imidazotriazine, pyrazinopyridazine, imidazopyridine, imidazopyrimidine, pyrazolopyridine, thiophene, benzothiophene, furan, pyran, cyclopentapyran, benzofuran, isobenzofuran, thiazole, isothiazole, benzothiazole, benzthiadiazole, isoxazole, furazane, oxazole, isoxazole, benzoxazole, oxadiazole, pyrazoloxazole, imidazothiazole, thienofuran, furopyrrole, pyridoxazine, etc.

In this specification, the “6-membered unsaturated hydrocarbon ring” refers to a benzene ring which may be partially hydrogenated, and the “6-membered unsaturated hetero ring containing 1 to 6 nitrogen atoms” refers to a pyridine ring which may be partially hydrogenated.

In this specification, the “5-membered hetero ring containing 1 or 2 nitrogen atoms” refers to pyrrole, pyrazole or imidazole, each of which may be partially hydrogenated.

The phrase “which may have a substituent group” in the definition of ring A used in this specification, the “substituent group” preferably means 1 to 3 substituent groups, and when a plurality of substituent groups are present, the definitions of the substituent groups are independent. Preferable examples of the “substituent group” include (1) an amino group which may be substituted with a lower alkyl group containing 1 to 6 carbon atoms or a lower cycloalkyl group containing 3 to 8 carbon atoms, (2) a lower alkyl group containing 1 to 6 carbon atoms, (3) a lower alkoxy group containing 1 to 6 carbon atoms, (4) hydroxyl group, (5) nitro group, (6) mercapto group, (7) cyano group, (8) a lower alkylthio group containing 1 to 6 carbon atoms, (9) ahalogeno group, (10) -a-b (wherein a isa single bond, —(CH₂)ₖ-, —O— (CH₂)ₖ—, —S—(CH₂)ₖ— or —N(R³)—(CH₂)ₖ— (wherein k is an integer of 1 to 5; and R³represents hydrogen atom or a lower alkyl group containing 1 to 6 carbon atoms); and b is —CH₂-d (wherein d represents an amino group which may be substituted with a lower alkyl group containing 1 to 6 carbon atoms, or a halogeno group, hydroxyl group, a lower alkylthio group containing 1 to 6 carbon atoms, cyano group or a lower alkoxy group containing 1 to 6 carbon atoms)), (11) -a-e-f (wherein a has the same meaning as defined above; e represents —SO— or —SO₂—; and f represents an amino group which may be substituted with a lower alkyl group containing 1 to 6 carbon atoms or a lower alkoxy group containing 1 to 6 carbon atoms, a lower alkyl group containing 1 to 6 carbon atoms, trifluoromethyl group, —(CH₂)ₘ-b or —N(R^{4)—(CH}₂)ₘ-b (wherein b has the same meaning as defined above; R⁴ represents a hydrogen atom or a lower alkyl group containing 1 to 6 carbon atoms; and m is an integer of 1 to 5)), (12) -a-g-h (wherein a has the same meaning as defined above; g represents —CO— or —CS—; and h represents an optionally substituted amino group, hydroxyl group, a lower alkyl group containing 1 to 6 carbon atoms, a lower alkoxy group containing 1 to 6 carbon atoms, —(CH₂)ₙ-b or —N(R⁵)—(CH₂)ₙ-b (wherein b has the same meaning as defined above; R⁵represents hydrogen atom or a lower alkyl group containing 1 to 6 carbon atoms; and n is an integer of 1 to 5)), (13) -a-N(R⁶)-g-i (wherein a and g have the same meanings as defined above; R⁶ represents hydrogen atom or a lower alkyl group containing 1 to 6 carbon atoms; i represents hydrogen atom, a lower alkoxy group containing 1 to 6 carbon atoms or f (wherein f has the same meaning as defined above)), (14) -a-N(R⁷)-e-f (wherein a, e and f have the same meanings as defined above; and R⁷ represents hydrogen atom or a lower alkyl group containing 1 to 6 carbon atoms), and (15) —(CH₂)p-j-(CH₂)q-b (wherein j is o or S; b has the same meaning as defined above; and p and q independently represent an integer of 1 to 5).

When an amino group in the substituent group is substituted with 2 alkyl groups, the alkyl groups may be combined together via a linkage to form a 5- or 6-membered nitrogen-containing ring.

When the ring A is a nitrogen-containing hetero ring having hydroxyl group or mercapto group as a substituent group, the substituent group may be an oxo or thioxo group by a resonance structure.

In the phrase “which may have a substituent group” in the definition of rings B and C used in this specification, the “substituent group” preferably means 1 or 2 substituent groups, and when a plurality of substituent groups are present, the definitions of the substituent groups are independent. The substituent groups on the rings B and C are also independent. Preferable examples of the “substituent group” include halogeno group, cyano group, a lower alkyl group containing 1 to 6 carbon atoms, a lower alkoxy group containing 1 to 6 carbon atoms, hydroxyl group, oxo group, —CO-r (wherein r represents hydrogen atom, an amino group which may be substituted with a lower alkyl group containing 1 to 6 carbon atoms, a lower alkyl group containing 1 to 6 carbon atoms, a lower alkoxy group containing 1 to 6 carbon atoms or hydroxyl group), an amino group which may be substituted with a lower alkyl group containing 1 to 6 carbon atoms and trifluoromethyl group.

The “lower alkyl group” used in this specification refers to an alkyl group containing 1 to 6 carbon groups, and preferable examples include linear or branched alkyl groups such as methyl group, ethyl group, n-propyl group, iso-propyl group, n-butyl group, iso-butyl group, sec-butyl group, tert-butyl group, n-pentyl group, 1,1-dimethylpropyl group, 1,2-dimethylpropyl group, 2,2-dimethylpropyl group, 1-ethylpropyl group, 2-ethylpropyl group, n-hexyl group, 1-methyl-2-ethylpropyl group, 1-ethyl-2-methylpropyl group, 1,1,2-trimethylpropyl group, 1-propylpropyl group, 1-methylbutyl group, 2-methylbutyl group, 1,1-dimethylbutyl group, 1,2-dimethylbutyl group, 2,2-dimethylbutyl group, 1,3-dimethylbutyl group, 2,3-dimethylbutyl group, 2-ethylbutyl group, 2-methylpentyl group and 3-methylpentyl group, more preferablymethyl group, ethyl group, n-propyl group, iso-propyl group, n-butyl group, iso-butylgroupetc., stillmorepreferably methyl group, ethyl group, n-propyl group, iso-propyl group etc.

The “lower cycloalkyl group” used in this specification refers to a cycloalkyl group composed of 3 to 8 carbon atoms, and preferable examples include cyclopropyl group, cyclobutyl group, cyclopentyl group, cyclohexyl group, cycloheptyl group, cyclooctyl group, etc.

The “C₁₋₆ alkoxy group” used in this specification refers to an alkoxy group containing 1 to 6 carbon groups, and preferable examples thereof include methoxy group, ethoxy group, n-propoxy group, iso-propoxy group, sec-propoxy group, n-butoxy group, iso-butoxy group, sec-butoxy group, tert-butoxy group, n-pentyloxy group, iso-pentyloxy group, sec-pentyloxy group, n-hexoxy group, iso-hexoxy group, 1,1-dimethyl propyloxy group, 1,2-dimethyl propoxy group, 2,2-dimethyl propyloxy group, 2-ethyl propoxy group, 1-methyl-2-ethyl propoxy group, 1-ethyl-2-methylpropoxy group, 1,1,2-trimethylpropoxy group, 1,1,2-trimethylpropoxy group, 1,1-dimethylbutoxy group, 1,2-dimethylbutoxy group, 2,2-dimethylbutoxy group, 2,3-dimethylbutyloxy group, 1,3-dimethylbutyloxy group, 2-ethylbutoxy group, 1,3-dimethylbutoxy group, 2-methylpentoxy group, 3-methylpentoxy group, hexyloxy group, etc., most preferably methoxy group and ethoxy group.

The “halogeno group” used in this specification refers to a group corresponding to a halogen atom, and includes fluoro group, chloro group, bromo group and iodo group, and preferable examples include fluoro group, chloro group and bromo group.

Preferable examples of the compound (I) contained in the medicinal composition according to the present invention are not particularly limited, but the most preferable compound is N-(3-chloro-1H-indol-7-yl)-4-sulfamoylbenzenesulfonamide or a pharmacologically acceptable salt thereof.

In this specification, N-(3-chloro-1H-indol-7-yl)-4-sulfamoylbenzenesulfonamide and E7070 refer to a compound represented by the formula (II):

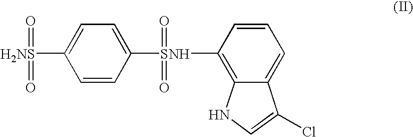

Irinotecan hydrochloride trihydrate (CPT-11; [1,4′-Bipiperidine]-1′-carboxylicacid (S)-4,11-diethyl-3,4,12,14-tetrahydro-4-hydroxy-3,14-dioxo-1H-pyrano-[3′,4′:6,7]-indolizino[1,2-b]quinolin-9-yl ester Hydrochloride trihydrate) in this specification refers to a compound represented by the formula:

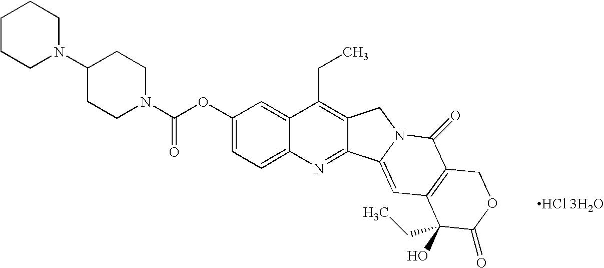

Mitomycin C (MMC; [1aS-(1aα,8β,8aα,8bα)]-6-amino-8-[[(aminocarbonyl)oxy]methyl]1,1a,2,8,8a,8b-hexahydro-8a-methoxy-5-methylazirino[2′,3′: 3,4]pyrrolo[1,2-a]indole-4,7-dione)) in this specification refers to a compound represented by the formula:

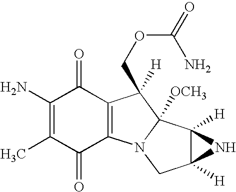

5-Fluorouracil (5-FU (5-fluoro-2,4-(1H,3H)-pyrimidinedione)) in this specification refers to a compound represented by the formula:

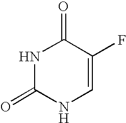

Cisplatin (CDDP (cis-diamminedichloroplatinum)) in this specification refers to a compound represented by the formula:

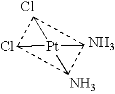

Gemcitabine hydrochloride (2′-deoxy-2′,2′-difluoro-cytidine hydrochloride) in this specification refers to a compound represented by the formula:

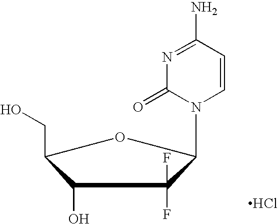

Doxorubicin (adryamicin; (10-[(3-amino-2,3,6-trideoxy-α-L-lyxo-hexopyranosyl)oxy]-7,8,9,10-tetrahydro-6,8,11-trihydroxy-8-(hydroxyacetyl)-1-methoxy-(8S-cis)-5,12-naphthacenedione) in this specification refers to a compound represented by the formula:

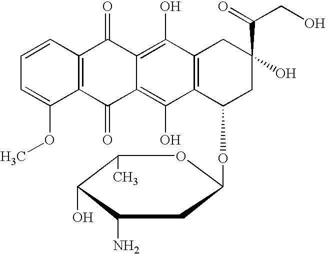

Taxol (paclitaxel; (2R,5R,7S,10R,13S)-10,20-bis-(acetoxy)-2-benzoyloxy-1,7-dihydroxy-9-oxo-5,20-epoxytax-1 1-en-13-yl (3S)-3-benzoylamino-3-phenyl-D-lactose) in this specification refers to a compound represented by the formula:

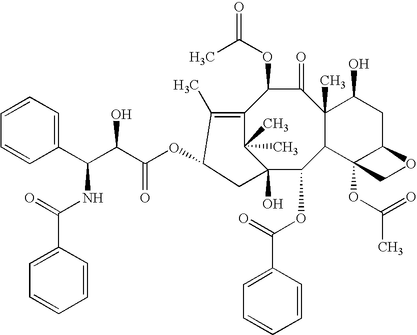

Carboplatin (cis-diammine (1,1-cyclobutanedi-carboxylato)platinum) in this specification refers toa compound represented by the formula:

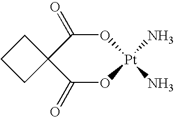

Oxaliplatin (oxalato (1R, 2R-cyclohexanediamine) platinum) in this specification refers to a compound represented by the formula:

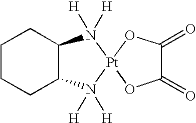

Capecitabine (N-4-pentyloxycarbonyl-5′-deoxy-5-fluorocytidine) in this specification refers to a compound represented by the formula:

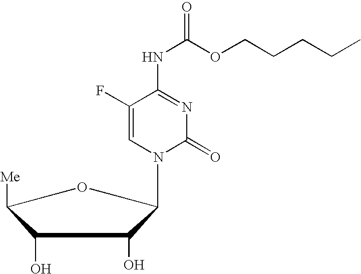

The type of the “salts” used in this specification is not particularly limited, and examples thereof include additive salts of inorganic acids such as hydrochloride, sulfate, carbonate, bicarbonate, hydrobromate, hydroiodate etc.; additive salts of organic carboxylic acids such as acetate, maleate, lactate, tartarate, trifluoroacetate etc.; additive salts of organic sulfonic acid such as methanesulfonate, hydroxymethanesulfonate, hydroxyethanesulfonate, benzenesulfonate, toluenesulfonate, taurine salt etc.; additive salts of amines such as trimethylamine salt, triethylamine salt, pyridine salt, procaine salt, picoline salt, dicyclohexylamine salt, N,N′-dibenzylethylene diamine salt, N-methylglucamine salt, diethanolamine salt, triethanolamine salt, tris(hydroymethylamino)methane salt, phenethylbenzylamine salt etc.; and additive salts of amino acids such as arginine salt, lysine salt, serine salt, glycine salt, aspartate, glutamate, etc.

The above-mentioned compound (I) can be produced according tothemethods described in e.g. JP-A7-165708, WO95/07276 etc., or its analogous method.

N-(3-Chloro-1H-indol-7-yl)-4-sulfamoylbenzene-sulfonamide (E7070) can be synthesized according to a known method (WO95/07276 or Example 19 in JP-A 7-165708) or its analogous method.

CPT-11, mitomycin C, 5-fluorouracil, cisplatin, gemcitabine hydrochloride, doxorubicin and taxol are known compounds and can be produced in known methods or their analogous methods.

Carboplatin, oxaliplatin and capecitabine are known compounds and can be produced in known methods or their analogous methods.

The starting compound and various reagents in the production processes may have formed salts or hydrates which vary depending on the starting material, the solvent used etc., and are not particularly limited so long as the reaction is not inhibited. Also, the solvent used varies depending on the starting material, reagents etc., and is not particularly limited insofar as it is inert to the reaction and dissolves the starting material to a certain degree. When the desired compound is obtained in a free form, it can be converted in a usual manner into a pharmaceutically acceptable salt. Further, the resultant various isomers (for example, geometric isomer, optical isomer based on asymmetric carbon, rotational isomer, stereoisomer, tautomer, etc.) can be purified and isolated by usual separating means, for example, re-crystallization, diastereomer salt method, enzyme resolution method, and various kinds of chromatography (for example, thin-layer chromatography, column chromatography, gas chromatography, etc.).

The “anticancer drug” in this specification means a drug used against tumors, particularly malignant tumors. The cancer disease against which the medicinal composition or the anticancer drug according to the present invention is effective includes, for example, brain cancer, head and neck cancer, cancer of the esophagus, cancer of the stomach, cancer of the colon, hepatoma, pancreatic cancer, lung cancer, breast cancer, skin cancer, ovarian cancer, prostatic cancer, renal cancer, bladder cancer, lymphoma, leukemia, etc. The medicinal composition or the anticancer drug according to the present invention is useful for treating or preventing cancer diseases in mammalians (e.g., humans, mice, rats, guinea pigs, rabbits, dogs, horses, monkeys, etc.), particularly for treating or preventing cancer diseases in humans.

In the present invention, “the medicinal composition combined with” and “the medicinal composition comprising” refer respectively to a “medicinal composition” comprising two or more active ingredients or medicinal compositions combined therein, and the “medicinal composition” may be prepared and administered as a single preparation comprising two or more active ingredients, or two or more medicinal compositions are separately prepared and simultaneously administered, or two or more medicinal compositions are separatelyprepared, and one of the compositions is administered, and after a predetermined time, the other composition may be administered. The “medicinal composition” in this invention encompasses any medicinal compositions in these administration forms. Further, the proportion of preferable ingredients in two or more medicinal compositions to be combined is not particularly limited and may be suitably selected. The proportion of the sulfone compound (I) and the substance (III) in the combined preparation is selected such that on the basis of the total dose for 3 weeks in clinical use, 1% by weight of the sulfone compound (I) such as E7070 or a salt thereof is combined with 0.26 to 1.0% by weight of irinotecan hydrochloride trihydrate or a salt thereof, 0.017 to 0.068% by weight of mitomycin C or a salt thereof, 3.7 to 15% by weight of 5-fluorouracil or a salt thereof, 0.071 to 0.29% by weight of cisplatin or a salt thereof,. 2.1 to 8.6% by weight of gemcitabine hydrochloride or a salt thereof, 0.042 to 0.17% by weight of doxorubicin or a salt thereof (preferably hydrochloride), or 0.15 to 0.60% by weight of taxol or a salt thereof.

One preferable example of the “medicinal composition” according to the present invention is a medicinal composition comprising N-(3-chloro-1H-indol-7-yl)-4-sulfamoylbenzenesulfonamide (E7070) or a salt thereof, combined with at least one substance selected from (1) irinotecan hydrochloride trihydrate; (2) mitomycin C; (3) 5-fluorouracil; (4) cisplatin; (5) gemcitabine hydrochloride; (6) doxorubicin; (7) taxol; (8) carboplatin; (9) oxaliplatin; (10) capecitabine; and (11) a salt of the above-mentioned (1) to (10), and more preferable examples include the following medicinal compositions:

(a) a medicinal composition comprising E7070 or a salt thereof, combined with irinotecan hydrochloride trihydrate or a salt thereof;

(b) a medicinal composition comprising E7070 or a salt thereof, combined with mitomycin C or a salt thereof;

(c) a medicinal composition comprising E7070 or a salt thereof, combined with 5-fluorouracil or a salt thereof;

(d) a medicinal composition comprising E7070 or a salt thereof, combined with cisplatin or a salt thereof;

(e) a medicinal composition comprising E7070 or a salt thereof, combined with gemcitabine hydrochloride or a salt thereof;

(f) a medicinal composition comprising E7070 or a salt thereof, combined with doxorubicin or a salt thereof;

(g) a medicinal composition comprising E7070 or a salt thereof, combined with taxol or a salt thereof;

(h) a medicinal composition comprising E7070 or a salt thereof,. combined with irinotecan hydrochloride trihydrate or a salt thereof and mitomycin C or a salt thereof;

(i) a medicinal composition comprising E7070 or a salt thereof, combined with irinotecan hydrochloride trihydrate or a salt thereof and 5-fluorouracil or salt thereof;

(j) a medicinal composition comprising E7070 or a salt thereof, combined with irinotecan hydrochloride trihydrate or a salt thereof and cisplatin or a salt thereof;

(k) a medicinal composition comprising E7070 or a salt thereof, combined with mitomycin C or a salt thereof and 5-fluorouracil or a salt thereof;

(l) a medicinal composition comprising E7070 or a salt thereof, combined with mitomycin C or a salt thereof and cisplatin or a salt thereof;

(m) a medicinal composition comprising E7070 or a salt thereof, combined with 5-fluorouracil or a salt thereof and cisplatin or a salt thereof;

(n) a medicinal composition comprising E7070 or a salt thereof, combined with mitomycin C or a salt thereof, 5-fluorouracil or a salt thereof and cisplatin or a salt thereof;

(o) a medicinal composition comprising E7070 or a salt thereof, combined with irinotecan trihydrate or a salt thereof, 5-fluorouracil or a salt thereof and cisplatin or a salt thereof;

(p) a medicinal composition comprising E7070 or a salt thereof, combined with irinotecan hydrochloride trihydrate or a salt thereof, mitomycin C or a salt thereof and cisplatin or a salt thereof;

(q) a medicinal composition comprising E7070 or a salt thereof, combined with irinotecan hydrochloride trihydrate or a salt thereof, mitomycin C or a salt thereof and 5-fluorouracil or a salt thereof;

(r) a medicinal composition comprising E7070 or a salt thereof, combined with irinotecan hydrochloride trihydrate or a salt thereof, mitomycin C or a salt thereof, 5-fluorouracil or a salt thereof and cisplatin or a salt thereof;

(s) a medicinal composition comprising E7070 or a salt thereof, combined with irinotecan hydrochloride trihydrate or a salt thereof and gemcitabine hydrochloride or a salt thereof;

(t) a medicinal composition comprising E7070 or a salt thereof, combined with irinotecan hydrochloride trihydrate or a salt thereof and doxorubicin or a salt thereof;

(u) a medicinal composition comprising E7070 or a salt thereof, combined with irinotecan hydrochloride trihydrate or a salt thereof and taxol or a salt thereof;

(v) a medicinal composition comprising E7070 or a salt thereof, combined with mitomycin C or a salt thereof and gemcitabine hydrochloride or a salt thereof;

(w) a medicinal composition comprising E7070 or a salt thereof, combined with mitomycin C or a salt thereof and doxorubicin or a salt thereof;

(x) a medicinal composition comprising E7070 or a salt thereof, combined with mitomycin C or a salt thereof and taxol or a salt thereof;

(y) a medicinal composition comprising E7070 or a salt thereof, combined with 5-fluorouracil or a salt thereof and gemcitabine hydrochloride or a salt thereof;

(z) a medicinal composition comprising E7070 or a salt thereof, combined with 5-fluorouracil or a salt thereof and doxorubicin or a salt thereof;

(aa) a medicinal composition comprising E7070 or a salt thereof, combined with 5-fluorouracil or a salt thereof and taxol or a salt thereof;

(bb) a medicinal composition comprising E7070 or a salt thereof, combined with cisplatin or a salt thereof and gemcitabine hydrochloride or a salt thereof;

(cc) a medicinal composition comprising E7070 or a salt thereof, combined with cisplatin or a salt thereof and doxorubicin or a salt thereof;

(dd) a medicinal composition comprising E7070 or a salt thereof, combined with cisplatin or a salt thereof and taxol or a salt thereof;

(ee) a medicinal composition comprising E7070 or a salt thereof, combined with gemcitabine hydrochloride or a salt thereof and doxorubicin or a salt thereof;

(ff) a medicinal composition comprising E7070 or a salt thereof, combined with gemcitabine hydrochloride or a salt thereof and taxol or a salt thereof;

(gg) a medicinal composition comprising E7070 or a salt thereof, combined with doxorubicin or a salt thereof and taxol or a salt thereof;

(ee) a medicinal composition comprising E7070 or a salt thereof, combined with gemcitabine hydrochloride or a salt thereof and doxorubicin or a salt thereof;

(ff) a medicinal composition comprising E7070 or a salt thereof, combined with gemcitabine hydrochloride or a salt thereof and taxol or a salt thereof;

(gg) a medicinal composition comprising E7070 or a salt thereof, combined with doxorubicin or a salt thereof and taxol or a salt thereof;

(hh) a medicinal composition comprising E7070 or a salt thereof, combined with mitomycin C or a salt thereof and 5-fluorouracil or a salt thereof and cisplatin or a salt thereof;

(ii) a medicinal composition comprising E7070 or a salt thereof, combined with mitomycin C or a salt thereof, 5-fluorouracil or a salt thereof and gemcitabine hydrochloride or a salt thereof;

(jj) a medicinal composition comprising E7070 or a salt thereof, combined with mitomycin C or a salt thereof, 5-fluorouracil or a salt thereof and doxorubicin and a salt thereof;

(kk) a medicinal composition comprising E7070 or a salt thereof, combined with mitomycin C or a salt thereof, 5-fluorouracil or a salt thereof and taxol or a salt thereof;

(ll) a medicinal composition comprising E7070 or a salt thereof, combined with 5-fluorouracil or a salt thereof, cisplatin or a salt thereof and gemcitabine hydrochloride or a salt thereof;

(mm) a medicinal composition comprising E7070 or a salt thereof, combined with 5-fluorouracil or a salt thereof, cisplatin or a salt thereof and doxorubicin or a salt thereof;

(nn) a medicinal composition comprising E7070 or a salt thereof, combined with 5-fluorouracil or a salt thereof, cisplatin or a salt thereof and taxol or a salt thereof;

(oo) a medicinal composition comprising E7070 or a salt thereof, combined with cisplatin or a salt thereof, gemcitabine hydrochloride or a salt thereof and doxorubicin or a salt thereof;

(pp) a medicinal composition comprising E7070 or a salt thereof, combined with cisplatin or a salt thereof, gemcitabine hydrochloride or a salt thereof and taxol or a salt thereof;

(qq) a medicinal composition comprising E7070 or a salt thereof, combined with gemcitabine hydrochloride or a salt thereof, doxorubicin or a salt thereof and taxol or a salt thereof;

(rr) a medicinal composition comprising E7070 or a salt thereof, combined with carboplatin or a salt thereof;

(ss) a medicinal composition comprising E7070 or a salt thereof, combined with oxaliplatin or a salt thereof;

(tt) a medicinal composition comprising E7070 or a salt thereof, combined with capecitabine or a salt thereof; and

(uu) a medicinal composition comprising E7070 or a salt thereof, combined with 5-fluorouracil or a salt thereof and oxaliplatin or a salt thereof.

The ingredients described above can be used to constitute the medicinal composition of the invention, and for administration of the medicinal composition or the anticancer drug, the administration is not limited to simultaneous administration, and the respective ingredients can be administrated separately at predetermined intervals to increase their synergistic effect.

Specifically, the synergistic effect can be increased by a method for preventing or treating cancers, by administering E7070 or a salt thereof to a patient; and administering, after a predetermined time, at least one substance selected from (1) irinotecan hydrochloride trihydrate; (2) mitomycin C; (3) 5-fluorouracil; (4) cisplatin; (5) gemcitabine hydrochloride; (6) doxorubicin; (7) taxol; (8) carboplatin; (9) oxaliplatin; (10) capecitabine; and (11) a salt of the above-mentioned (1) to (10) to the patient, or by a method for preventing or treating cancers, by administering at least one substance selected from (1) irinotecan hydrochloride trihydrate; (2) mitomycin C; (3) 5-fluorouracil; (4) cisplatin; (5) gemcitabine hydrochloride; (6) doxorubicin; (7) taxol; (8) carboplatin; (9) oxaliplatin; (10) capecitabine; and (11) a salt of the above-mentioned (1) to (10) to a patient; and administering, after a predetermined time, E7070 or a salt thereof to the patient, and the present invention encompasses such methods for preventing or treating.

The synergistic antitumor effect can also be achieved by using a pharmaceutical kit for administering effective amounts of E7070 or a salt thereof and at least one substance selected from (1) irinotecan hydrochloride trihydrate; (2) mitomycin C; (3) 5-fluorouracil; (4) cisplatin; (5) gemcitabine hydrochloride; (6) doxorubicin; (7) taxol; (8) carboplatin; (9) oxaliplatin; (10) capecitabine; and (11) a salt of the above-mentioned (1) to (10) simultaneously or separately to a patient, for the purpose of administration of the respective ingredients at predetermined intervals as in the above-mentioned methods for preventing or treating. The present invention encompasses such a pharmaceutical kit. For example, mention is made of a pharmaceutical kit wherein the ingredients, each being packed in a small vessel, are packed in one box.

When the medicinal composition of the present invention is used as a pharmaceutical preparation, the administration form is not particularly limited, and it is administered orally or parenterally. It is useful for treatment and prevention in mammalians (e.g., humans, mice, rats, guinea pigs, rabbits, dogs, horses, monkeys, etc.), particularly in humans. The administration dose varies depending on the severity of symptoms, the age, sex, body weight and chemical sensitivity of the patient, administration form, administration time, administration intervals, the properties, prescription and type of the pharmaceutical preparation, and the type of active ingredient, and is not particularly limited. For example, E7070 or a salt thereof is given daily in one portion or in divided portions into a man in a dose of usually about 5 to 6000 mg, preferably about 50 to 4000 mg, more preferably 100 to 3000 mg. The daily doses of the other ingredients are usually as follows: irinotecan hydrochloride trihydrate, 40 to 150 mg/m²; mitomycin C, 2 to 40 mg; 5-fluorouracil, 5 to 20 mg/kg; cisplatin, 10 to 90 mg/m²; gemcitabine hydrochloride, 500 to 1200 mg/m²; doxorubicin hydrochloride, 10 to 60 mg; taxol, 135 to 210 mg/m²; carboplatin, 300 to 400 mg/m²; oxaliplatin, 85 to 130 mg/m²; and capecitabine, 1900 to 2500 mg/m², but these are standard doses in the case of administering a single drug. In the present invention, the dose can be suitably changed depending on the constitution of the ingredient. For example, the daily dose of the respective ingredients for an adult can be determined usually in the range of 1 to 6000 mg, preferably about 10 to 1000 mg, more preferably 20 to 300 mg.

The medicinal composition according to the invention can be prepared by using the active ingredients as they are or mixing them with pharmacologically acceptable carriers known per se by a conventional method. Preferable preparation forms include injections, tablets, powders, fine granules, granules, coated tablets, capsules, syrups, troches, inhalations, suppositories, ointments, eye ointments, eye drops, nose drops, ear drops, poultices and lotions. To prepare these pharmaceutical preparations, ordinarily used fillers, binders, lubricants, coloring agents, flavoring agents and, if necessary, stabilizers, emulsifiers, absorption promoters and surfactants can be used. Ingredients used generally as starting materials for pharmaceutical preparations can be blended in a usual manner for manufacturing.

As the above-mentioned preparation components, for example, include animal or vegetable oils such as soybean oil, beef tallow or synthetic glyceride; hydrocarbons such as liquid paraffin, squalane or solid paraffin; ester oils such as octyldodecyl myristate or isopropyl myristate; higher alcohols such as cetostearyl alcohol or behenyl alcohol; silicon resin; silicon oil; surfactants such as polyoxyethylene fatty acid ester, sorbitan fatty acid ester, glycerol fatty acid ester, polyoxyethylenesorbitan fatty acid ester, polyoxyethylene hydrogenated castor oil or polyoxyethylene/polyoxypropylene block copolymers; water-soluble polymers such as hydroxyethyl cellulose, polyacrylic acid, carboxyvinyl polymers, polyethylene glycol, polyvinylpyrrolidone or methyl cellulose; alcohols such as ethanol or isopropanol; polyhydric alcohols such as glycerol, propylene glycol, dipropylene glycol or sorbitol; sugars such as glucose or sucrose; inorganic powders such as silicic anhydride, aluminum magnesium silicate or aluminum silicate; and purified water. For pH adjustment, inorganic acids such as hydrochloric acid or phosphoric acid, alkali metal salts of inorganic acids such as sodium phosphate, inorganic bases such as sodium hydroxide, organic acids such as lower fatty acids, citric acid or lactic acid, alkali metal salts of organic acids such as sodium citrate or sodium lactate, organic bases such as arginine or ethanolamine, etc. can be used. If necessary, a preservative, an antioxidant, etc. can be added.

For example, when a solid preparation for oral administration is prepared, filler and, if necessary, binder, disintegrating agent, lubricant, coloring agent, flavoring agent etc. are added to the main ingredient, followed by subjecting to a common method to make into tablets, coated tablets, granules, fine granules, powders, capsules etc.

Examples of the filler are lactose, corn starch, sucrose, glucose, sorbitol, crystalline cellulose and silicon dioxide; examples of the binder are polyvinyl alcohol, ethyl cellulose, methyl cellulose, gum arabic, hydroxypropyl cellulose and hydroxypropyl methyl cellulose; examples of the lubricant are magnesium stearate, talc and silica; examples of the coloring agent are those which are allowed to add to pharmaceuticals; and examples of the flavoring agents are cacao powder, menthol, aromatic, peppermint oil, borneol and cinnamon powder. It is of course no problem that such tablets and granules are appropriately coated with a sugar coat, gelatin coat or others if necessary.

In preparing injections, a pH adjusting agent, a buffer, a suspending agent, a solubilizer, a stabilizer, an isotonizing agent, a preservative etc. are added, if necessary, to the main ingredient, followed by subjecting to a conventional method to make into injections for intravenous, subcutaneous or intra muscular administration. At that time, it may be made into a freeze-dried product by a common method if necessary.

Examples of the suspending agent are methyl cellulose, polysorbate 80, hydroxyethyl cellulose, gum arabic, tragacanth powder, sodium carboxymethyl cellulose and polyoxyethylene sorbitan monolaurate.

Examples of the solubilizer are polyoxyethylene hydrogenated castor oil, polysorbate 80, nicotinamide, polyoxyethylene sorbitan monolaurate, macrogol and castor oil fatty acid ethyl ester.

Examples of the stabilizer are sodium sulfite and sodium metasulfite. Examples of the preservative are methyl para-hydroxybenzoate, ethylpara-hydroxybenzoate, sorbicacid, phenol, cresol and chlorocresol.

According to the present invention, there can be provided a novel combined medicinal composition exhibiting an antitumor activity even on cancers against which conventional anticancer drugs are not sufficiently effective. The medicinal composition according to the present invention is useful for treating or preventing brain cancer, head and neck cancer, cancer of the esophagus, cancer of the stomach, cancer of the colon, hepatoma, pancreatic cancer, lung cancer, breast cancer, skin cancer, ovarian cancer, prostatic cancer, renal cancer, bladder cancer, lymphoma, leukemia, etc. Administration of smaller amounts of the combined active ingredients in the composition of the present invention, as compared with administration of large amounts of each single ingredient, hardly causes the side effect of each ingredient, can reduce the side effects of the whole ingredients, and can reduce the cost burden which is caused by using a large amount of an expensive drug for a long time, by simultaneously using inexpensive drugs having a relatively strong effect.

### BRIEF DESCRIPTION OF THE DRAWINGS

FIG. 1 is a graph showing the synergy of E7070 and CPT-11. The relative tumor volume (RTV) is shown on the ordinate, and the number of days after administration was initiated is shown on the axis;

FIG. 2 is a graph showing the synergy of E7070 and MMC. The relative tumor volume (RTV) is shown on the ordinate, and the number of days after administration was initiated is shown on the axis;

Each of FIGS. 3(1), 3(2) and 3(3) is a graph showing the synergy of E7070 and CPT-11. The relative tumor volume (RTV) is shown on the ordinate, and the number of days after administration was initiated is shown on the axis;

FIG. 3(1) shows simultaneous administration of E7070 and CPT-11, FIG. 3(2) shows administration of E7070 and subsequent administration CPT-11, and FIG. 3(3) shows administration of CPT-11 and subsequent administration of E7070;

FIG. 4 is a graph showing the synergy of E7070 and 5-FU. The relative tumor volume (RTV) is shown on the ordinate, and the number of days after administration was initiated is shown on the axis; and

FIG. 5 is a graph showing the synergy of E7070 and CDDP. The relative tumor volume (RTV) is shown on the ordinate, and the number of days after administration was initiated is shown on the axis.

FIG. 6 is a graph showing the effect of the combination of E7070 and capecitabine. The relative tumor weight (above) and the relative body weight (below) are shown on the ordinate, and the number of days after administration was initiated is shown on the axis.

FIG. 7 is a graph showing the criteria of the isobologram. The dose of B is shown on the ordinate, and the dose of A is shown on the axis.

FIG. 8 is a graph showing the criteria of the isobologram analysis of the combination of E7070 with cisplatin in HCT15 cells. The dose of cisplatin is shown on the ordinate, and the dose of E7070 is shown on the axis.

FIG. 9 is a graph showing the criteria of the isobologram analysis of the combination of E7070 with carboplatin in HCT15 cells. The dose of carboplatin is shown on the ordinate, and the dose of E7070 is shown on the axis.

FIG. 10 is a graph showing the criteria of the isobologram analysis of the combination of E7070 with oxaliplatin in HCT15 cells. The dose of oxaliplatin is shown on the ordinate, and the dose of E7070 is shown on the axis.

### EXAMPLES

Hereinafter, Examples and Test Examples are described to show the advantageous effect of the composition of the present invention, but these are illustrative, and in any case the present invention is not limited to the following specific examples.

As described above, the medicinal composition according to the invention, particularly a medicinal composition comprising E7070 or a salt thereof combined with at least one substance selected from (1) irinotecan hydrochloride trihydrate; (2) mitomycinC; (3) 5-fluorouracil; (4) cisplatin; (5) gemcitabine hydrochloride; (6) doxorubicin; (7) taxol; (8) carboplatin; (9) oxaliplatin; (10) capecitabine; and (11) a salt of the above-mentioned (1) to (10) exhibits an excellent antitumor activity due to the synergistic antitumor effect of each single drug, and the presence or absence of this synergy was examined according to a two-way ANOVA method (see the following literatures (i) to (iii): (i)Effects of 5-fluorouracil, leucovorin, and glucarate in rat colon-tumor explants. Cancer Chemother Pharmacol. 1992;30(1):25-30; (ii)Enhancement of vincristine cytotoxicity in drug-resistant cells by simultaneous treatment with onconase, an antitumor ribonuclease. J Natl Cancer Inst. 1996 Jun 5;88(11):747-53; (iii)Effects of growth hormone and testosterone on cortical bone formation and bone density in aged orchiectomized rats. Bone. 1999 May;24(5):491-7).

### Example 1

### Combined use of E7070 and CPT-11 in Human Colon Cancer HCT15 Xenograft Model

Human colon cancer strain HCT15 (purchased from ATCC) was cultured in RPMI1640 (containing 10%FBS) in a 5%CO₂gas incubator until it attained about 80% confluence, and the cells were harvested with trypsin-EDTA and suspended in Hanks balanced solution to prepare a suspension of 5×10⁷ cells/ml. 0.1 ml of the cell suspension was implanted subcutaneously in each nude mouse. When the average tumor volume reached 182 mm3 after the implantation, E7070 in a dose of 30 mg/kg/day and/or CPT-11 in a dose of 75 mg/kg/day were administered either alone or in combination. E7070was intravenously administered once per day for 5 days (first to fifth days), while CPT-11 was intravenously administered 3 times (once every 4 days, that is, on the first, fifth and ninth days). After the administration was initiated, the longer and shorter diameters of the tumor were measured at the frequency of 2 times/week with digital calipers (Mitsutoyo), and the tumor volume was calculated according to the following equation.

Tumor volume=longer diameter of tumor (mm)×shorter diameter of tumor (mm)²/2

The items for determination of antitumor effect were the following 2 items:

Tₓ₄: The time in days for tumor size to attain 4 folds of initial size.

Minimum relative tumor volume (mRTV): Minimum value of relative tumor volume (RTV*)

*: Tumor volume on n days after first treatment/tumor volume at the start of treatment.

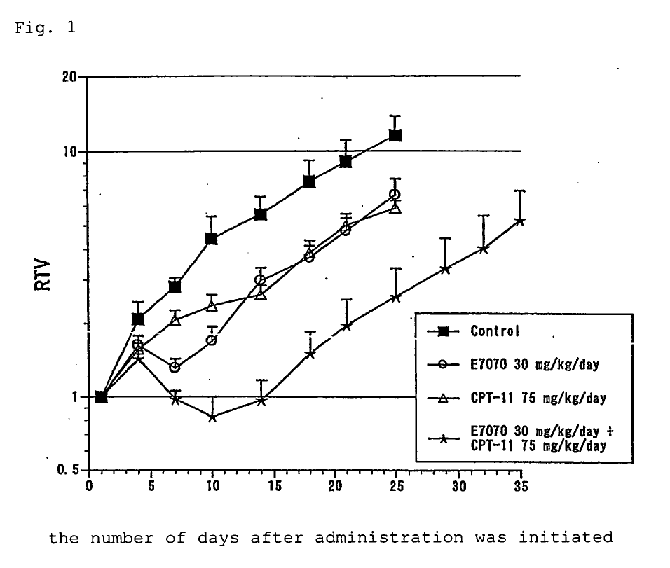

When the group treated with the combined drugs exhibited an antitumor effect superior to that of the group treated with the single drug and simultaneously a statistically significant interaction was recognized in two-way ANOVA, the antitumor effect was determined to be synergistic. Results are as shown in Table 1 and FIG. 1.

As is evident from Table 1 and FIG. 1, combined use of E7070 and CPT-11 permits their respective effects to be synergistically increased, indicating that a combined drug of E7070 and CPT-11 serves as an excellent anticancer agent.

### Example 2

### Combined use of E7070 and MMC in Human Colon Cancer HCT15 Xenograft Model

Human colon cancer strain HCT15 (purchased from ATCC) was cultured in RPMI1640 (containing 10% FBS) in a 5% CO₂gas incubator until it attained about 80% confluence, and the cells were harvested with trypsin-EDTA and suspended in Hanks balanced solution to prepare a suspension of 5×10⁷ cells/ml. 0.1 ml of the cell suspension was implanted subcutaneously in each nude mouse. When the average tumor volume reached 156 mm3 after the implantation, E7070 in a dose of 25 mg/kg/day and/or MMC in a dose of 4.19 mg/kg/day were administered either alone or in combination. E7070was intravenously administered once per day for 5 days (first to fifth days), while MMC was intravenously administered once (first day). After the administration was initiated, the longer and shorter diameters of the tumor were measured at the frequency of 2 times/week with digital calipers (Mitsutoyo), and the tumor volume was calculated according to the following equation.

Tumor volume=longer diameter of tumor (mm)×shorter diameter of tumor (mm)²/2

The items for determination of antitumor effect were the following 2 items:

Tₓ₄ The time in days for tumor size to attain 4 folds of initial size.

Minimum relative tumor volume (mRTV): Minimum value of relative tumor volume (RTV*)

*: Tumor volume on n days after first treatment/tumor volume at the start of treatment.

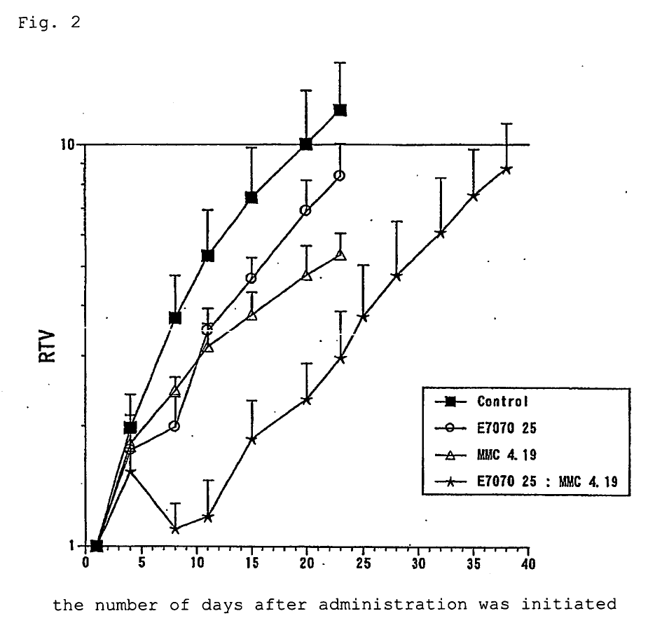

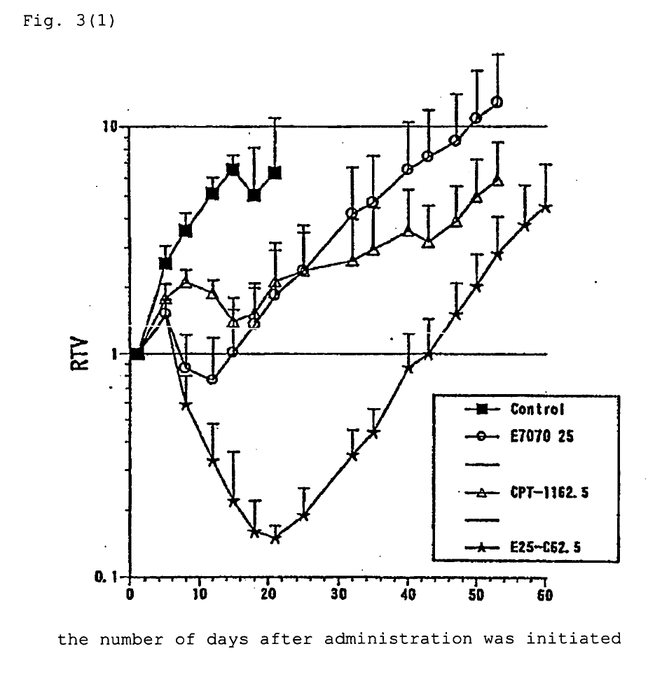

When the group treated with the combined drugs exhibited an antitumor effect superior to that of the group treated with the single drug and simultaneously a statistically significant interaction was recognized in two-way ANOVA, the antitumor effect was determined to be synergistic. Results are as shown in Table 2 and FIG. 2.

As is evident from Table 2 and FIG. 2, combined use of E7070 and MMC permits their respective effects to be synergistically increased, indicating that a combined drug of E7070 and MMC serves as an excellent anticancer agent.

### Example 3

### Combined use of E7070 and CPT-11 in Human Colon Cancer SW620 Xenograft Model

Human colon cancer strain SW620 (purchased from ATCC) was cultured in RPMI1640 (containing 10% FBS) in a 5% Co₂gas incubator until it attained about 80% confluence, and the cells were harvested with trypsin-EDTA and suspended in Hanks balanced solution to prepare a suspension of 5×10⁷ cells/ml. 0.1 ml of the cell suspension was implanted subcutaneously in each nude mouse. When the average tumor volume reached 226 mm³ after the implantation, E7070 in a dose of 25 mg/kg/day and/or CPT-11 in a dose of 62.5 mg/kg/day were administered either alone or in combination. E7070 alone was intravenously administered once per day for 5 days (first to fifth days), while CPT-11 alone was intravenously administered 3 times (once every 4 days, that is, on the first, fifth and ninth days). Administration in combination was performed in 3 schedules, that is, simultaneous administration (E7070 on the first to fifth days; CPT-11 on the first, fifth and ninth days), previous administration of E7070 (E7070 on the first to fifth days; CPT-11 on the sixth, tenth and fourteenth days) and previous administration of CPT-11 (E7070 on the tenth to fourteenth days; CPT-11 on the first, fifth and ninth days). After the administration was initiated, the longer and shorter diameters of the tumor were measured at the frequency of 2 times/week with digital calipers (Mitsutoyo), and the tumor volume was calculated according to the following equation.

Tumor volume=longer diameter of tumor (mm)×shorter diameter of tumor (mm)²/2

The items for determination of antitumor effect were the following 2 items:

Tₓ₄: The time in days for tumor size to attain 4 folds of initial size.

Minimum relative tumor volume (mRTV): Minimum value of relative tumor volume (RTV*)

*: Tumor volume on n days after first treatment/tumor volume at the start of treatment.

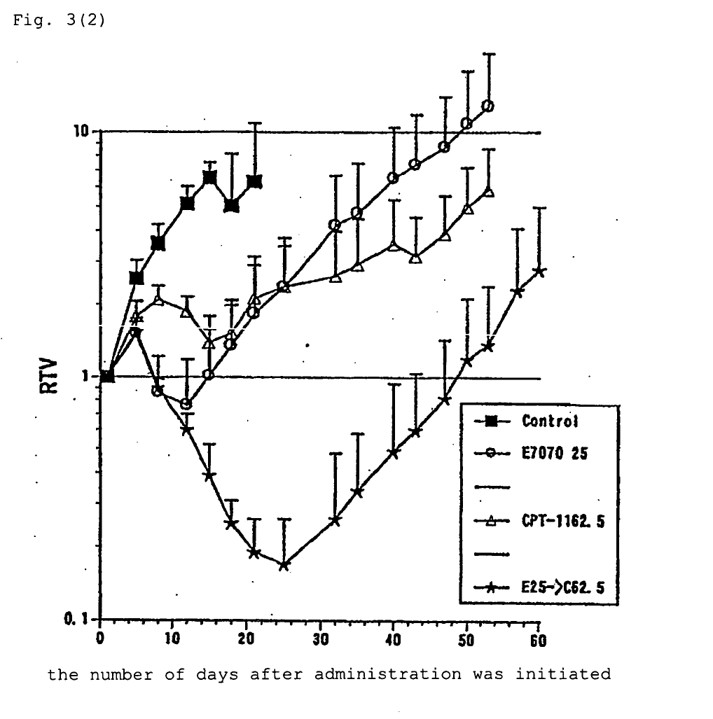

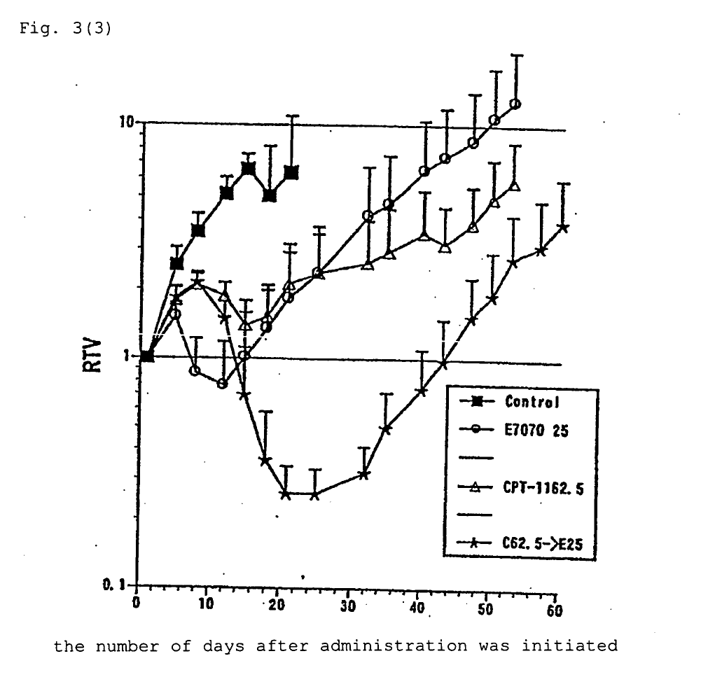

When the group treated with the combined drugs exhibited an antitumor effect superior to that of the group treated with the single drug and simultaneously a statistically significant interaction was recognized in two-way ANOVA, the antitumor effect was determined to be synergistic. Results are as shown in Table 3 and FIG. 3.

As is evident from Table 3 and FIG. 3, combined use of E7070 and CPT-11 permits their respective effects to be synergistically increased, indicating that a combined drug of E7070 and CPT-11 serves as an excellent anticancer drug. Further, E7070 and CPT-11 may be administered simultaneously, or one of the two may be administered after a predetermined time, and E7070 and CPT-11, whichever is administered first, exhibits a synergistic effect.

### Example 4

### Combined use of E7070 and 5-FU in Human Colon Cancer Colo320D. M. Xenograft Model

Human colon cancer strain Colo320D. M. (purchased from ATCC) was cultured in RPMI1640 (containing 10% FBS) in a 5 % CO₂ gas incubator until it attained about 80% confluence, and the cells were harvested with trypsin-EDTA and suspended in Hanks balanced solution to prepare a suspension of 8×10⁷ cells/ml. 0.1 ml of the cell suspension was implanted subcutaneously in each nude mouse. When the average tumor volume reached 169 MM3 after the implantation, E7070 in a dose of 30 mg/kg/day and/or 5-FU in a dose of 60 mg/kg/day were administered either alone or in combination. E7070 alone was intravenously administered per day for 5 days (first to fifth days), while 5-FU alone was intravenously administered 3 times (once every 4 days, that is, on the first, fifth and ninth days). Administration in combination was performed in 3 schedules, that is, simultaneous administration (E7070 on the first to fifth days; 5-FU on the first, fifth and ninth days), previous administration of E7070 (E7070 on the first to fifth days; 5-FU on the sixth, tenth and fourteenth days) and previous administration of 5-FU (E7070 on the tenth to fourteenth days; 5-FU on the first, firth and ninth days). After the administration was initiated, the longer and shorter diameters of the tumor were measured at the frequency of 2 times/week with digital calipers (Mitsutoyo), and the tumor volume was calculated according to the following equation. Tumor volume=longer diameter of tumor (mm)×shorter diameter of tumor (mm)²/2

The items for determination of antitumor effect were the following 2 items:

Tₓ₄: The time in days for tumor size to attain 4 folds of initial size.

Minimum relative tumor volume (mRTV): Minimum value of relative tumor volume (RTV*)

*: Tumor volume on n days after first treatment/tumor volume at the start of treatment.

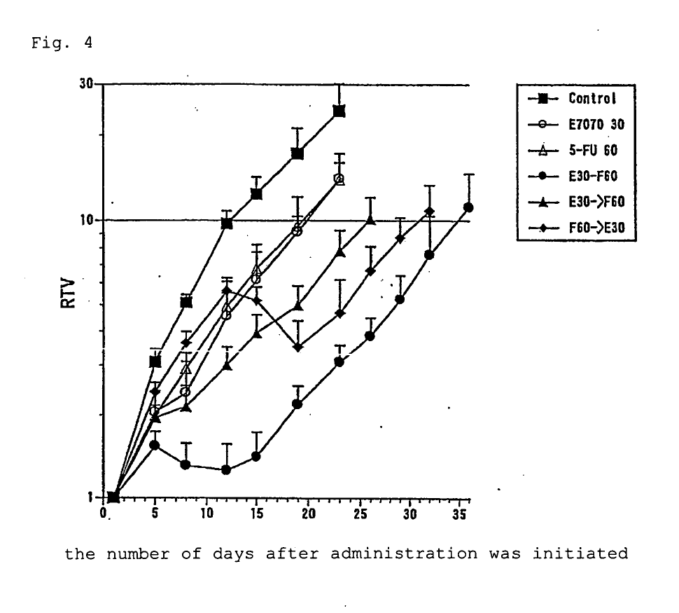

When the group treated with the combined drugs exhibited an antitumor effect superior to that of the group treated with the single drug and simultaneously a statistically significant interaction was recognized in two-way ANOVA, the antitumor effect was determined to be synergistic. Results are as shown in Table 4 and FIG. 4.

As is evident from Table 4 and FIG. 4, combined use of E7070 and 5-FU permits their respective effects to be synergistically increased, indicating that a combined drug of E7070 and 5-FU serves as an excellent anticancer agent.

### Example 5

### Combined use of E7070 and CDDP in Human non-small Cell Lung Cancer LU-99 Xenograft Model

Human non-small cell lung cancer LU-99 (purchased from Human Science Research Resources Bank) was cultured in RPMI1640 (containing 10% FBS) in a 5 % CO₂ gas incubator until it attained about 80% confluence, and the cells were harvested with trypsin-EDTA and suspended in Hanks balanced solution to prepare a suspension of 6.4×10⁷ cells/ml. 0.1 ml of the cell suspension was implanted subcutaneously in each nude mouse. When the average tumor volume reached 114 mm³ after the implantation, E7070 in a dose of 25 mg/kg/day and/or CDDP in a dose of 7.5 mg/kg/day were administered either alone or in combination. E7070 alone was intravenously administered once per day for 5 days (first to fifth days), while CDDP alone was intravenously administered once (first day). Administration in combination was performed in 3 schedules, that is, simultaneous administration (E7070 on the first to fifth days; CDDP on the first day), previous administration of E7070 (E7070 on the first to fifth days; CDDP on the sixth day) and previous administration of CDDP (E7070 on the second to sixth days; CDDP on the first day). After the administration was initiated, the longer and shorter diameters of the tumor were measured at the frequency of 2 times/week with digital calipers (Mitsutoyo), and the tumor volume was calculated according to the following equation.

Tumor volume=longer diameter of tumor (mm)×shorter diameter of tumor (mm)²/2

The items for determination of antitumor effect were the following 2 items:

Tₓ₄: The time in days for tumor size to attain 4 folds of initial size.

Minimum relative tumor volume (mRTV): Minimum value of relative tumor volume (RTV*)

*: Tumor volume on n days after first treatment/tumor volume at the start of treatment.

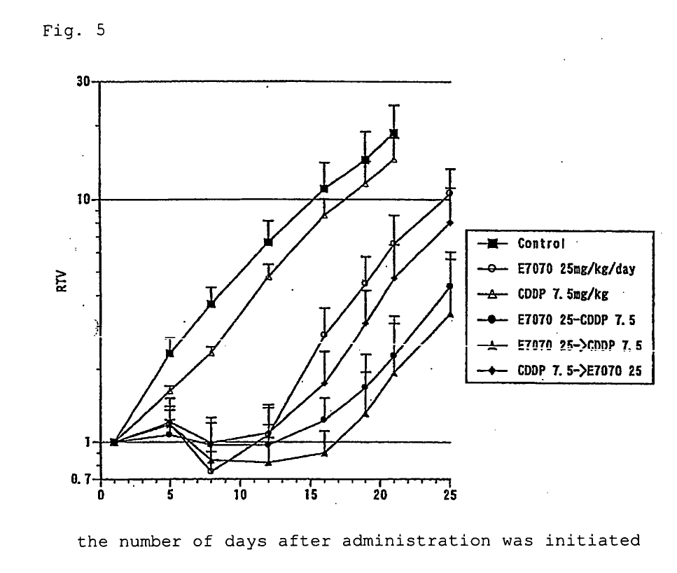

When the group treated with the combined drugs exhibited an antitumor effect superior to that of the group treated with the single drug and simultaneously a statistically significant interaction was recognized in two-way ANOVA, the antitumor effect was determined to be synergistic. Results are as shown in Table 5 and FIG. 5.

As is evident from Table 5 and FIG. 5, combined use of E7070 and CDDP permits their respective effects to be synergistically increased, indicating that a combined drug of E7070 and CDDP serves as an excellent anticancer agent.

### Example 6

### Evaluation of the Effect of Combined use of E7070 and Gemcitabine in Human Colon Cancer HCT15 Xenograft Model

Human colon cancer strain HCT15 (purchased from ATCC) was cultured in RPMI1640 (containing 10% FBS) in a 5% CO₂ gas incubator until it attained about 80% confluence, and the cells were harvested with trypsin-EDTA and suspended in Hanks balanced solution to prepare a suspension of 5×10⁷ cells/ml. 0.1 ml of the cell suspension was implanted subcutaneously in each nude mouse. When the average tumor volume reached 156 mm3 after the implantation, E7070 in a dose of 25 mg/kg/day and/or MMC in a dose of 4.19 mg/kg/day were administered either alone or in combination. E7070 was intravenously administered once per day for 5 days (first to fifth days), while CPT-11 was intravenously administered 4 times (once every 3 days, that is, on the first, fourth, seventh and tenth days). After the administration was initiated, the longer and shorter diameters of the tumor were measured at the frequency of 2 times/week with digital calipers (Mitsutoyo), and the tumor volume was calculated according to the following equation.

Tumor volume=longer diameter of tumor (mm)×shorter diameter of tumor (mm)²/2

The items for determination of antitumor effect were the following 2 items:

Tₓ₄: The time in days for tumor size to attain 4 folds of initial size.

Minimum relative tumor volume (mRTV): Minimum value of relative tumor volume (RTV*)

*: Tumor volume on n days after first treatment/tumor volume at the start of treatment.

When the group treated with the combined drugs exhibited an antitumor effect superior to that of the group treated with the single drug and simultaneously a statistically significant interaction was recognized in two-way ANOVA, the antitumor effect was determined to be synergistic.

### Example 7

### Combined use of E7070 and Capecitabine in Human Breast Cancer HBC4 Xenograft Model

Human breast cancer HBC4 (purchased from the Cancer Chemotherapy Center, Japan Foundation for Cancer Research (Tokyo, Japan)) was cultured in RPMI1640 (containing 10% FBS) in a 5% CO₂ gas incubator until it attained about 80% confluence, and the cells were harvested with trypsin-EDTA and suspended in Hanks balanced solution to prepare a suspension of 5×10⁷ cells/ml. 0.1 ml of the cell suspension was implanted subcutaneously in each nude mouse. When the average tumor volume reached 114 mm3 after the implantation, E7070 in a dose of 30.625 mg/kg/day and/or capecitabine in a dose of 1.3125 mmol/kg/day were administered either alone or in combination. E7070 alone was intravenously administered once per day for 5 days (first to fifth days), while capecitabine alone was intravenously administered once per day for 14 days (first to fourteenth days). Previous administration of E7070 (E7070 on the first to fifth days; capecitabine on the sixth to nineteenth days) was performed. After the administration was initiated, the longer and shorter dianometers of the tumor were measured at the frequency of 2 times/week with digital calipers (Mitsutoyo), and the tumor volume was calculated according to the following equation.

Tumor volume=longer diameter of tumor (mm)×shorter diameter of tumor (mm)²/2

The items for determination of antitumor effect were the following 2 items:

Tₓ₄: The time in days for tumor size to attain 4 folds of initial size.

Minimum relative tumor volume (mRTV): Minimum value of relative tumor volume (RTV*)

*: Tumor volume on n days after first treatment/tumor volume at the start of treatment.

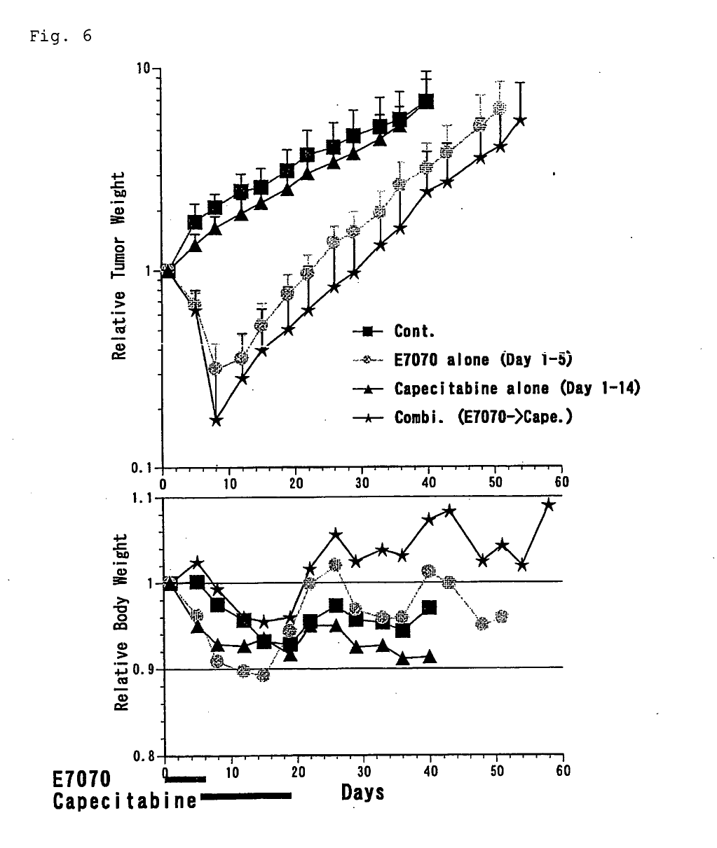

When the group treated with the combined drugs exhibited an antitumor effect superior to that of the group treated with the single drug and simultaneously a statistically significant interaction was recognized in two-way ANOVA, the antitumor effect was determined to be synergistic. Results are as shown in Table 6 and FIG. 6.

As is evident from Table 6 and FIG. 6, combined use of E7070 and capecitabine permits their respective effects to be additively increased, indicating that a combined drug of E7070 and capecitabine serves as an excellent anticancer agent.

As is evident from the above-mentioned experimental examples, the compositions of the present invention exhibit an excellent antitumor activity and are useful as an antitumor agent. In addition to the compounds used above, gemcitabine hydrochloride, doxorubicin and taxol were used and examined for their synergistic effect, indicating that they also exhibit an excellent antitumor activity and are useful as an antitumor agent. As the cancer type, cancers against which the respective ingredients are effective may be proposed, and since it varies depending on the constitution of the composition and further the synergistic effect in the present invention is higher than that of a single drug, the type of cancer is not particularly limited.

### Example 8

### Evaluation of the Effect of Combined use of E7070, Cisplatin, Carboplatin, and Oxaliplatin in Human Colon Cancer HCT15 Cells in Vitro

Rapidly growing Human colon cancer strain HCT15 cells(purchased from ATCC) were harvested, counted,and inoculated at appropriate concentrations (1,250 cells/well) into 96-well microplates. The following day (dayo), drugs were dissolved in DMSO (E7070), saline(cisplatin and carboplatin) or 5% glucose solution (oxaliplatin), diluted with culture medium at various concentrations, and then applied to triplicate culture wells.

In the case of simultaneous treatment, E7070 and cisplatin, carboplatin, or oxaliplatin were added at a time. After further 72-h incubation at 37° C. and 5% CO2, the amount of viable cells was determined by the MTT assay (day3).

In the case of sequential treatment (E7070 pretreatment), E7070 was added on day 0. After 24-h incubation, E7070 was washed out with fresh medium and then platinum was added (day1). After another 48-h incubation at 37° C. and 5% CO2, the amount of viable cells was determined by the MTT assay (day3).

In the case of sequential treatment (platinum pretreatment), platinum, that is, cisplatin, carboplatin or oxaliplatin was added on day 0. After 24-h incubation, platinum was washed out with fresh medium and then E7070 was added (day1). After another 48-h incubation at 37° C. and 5% CO2, the amount of viable cells was determined by the MTT assay (day3).

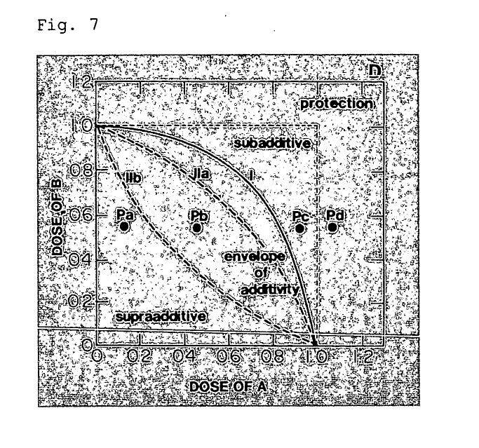

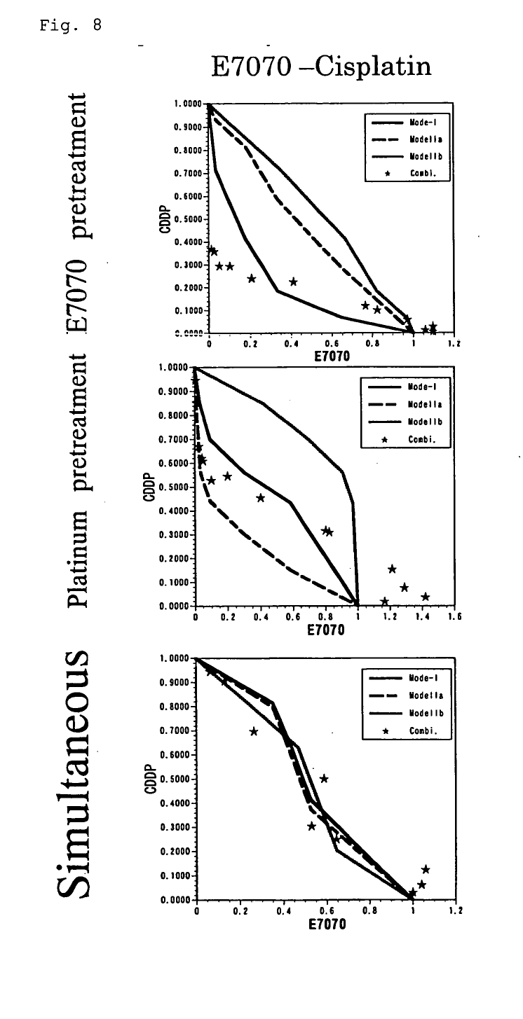

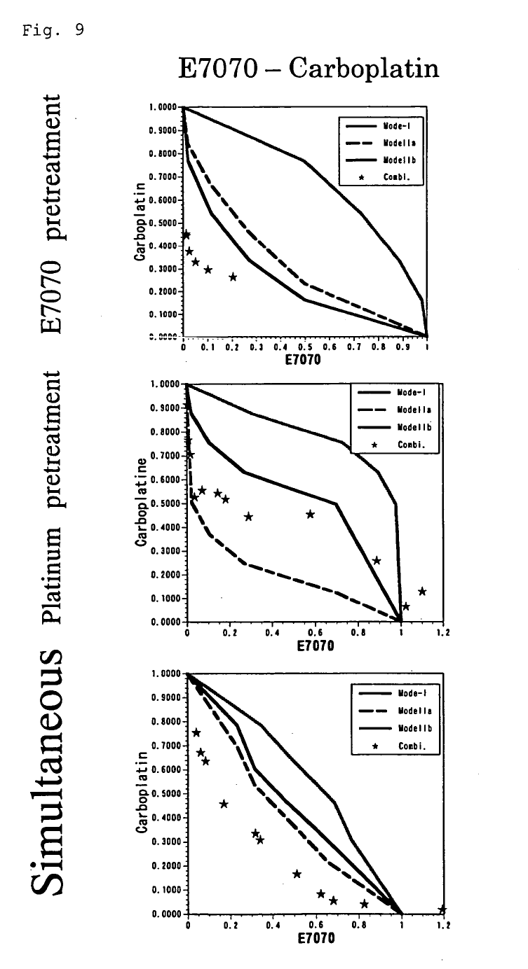

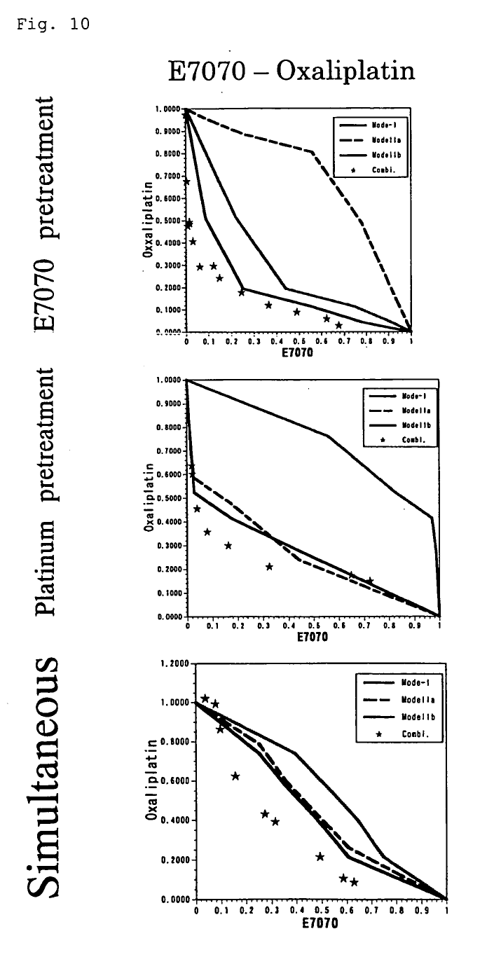

The effects of E7070 in combination with cisplatin, carboplatin or oxaliplatin were analyzed using the isobologram method of Steel and Peckam (Steel GG and Peckham M J: Exploitable mechanisms in combined radiotherapy-chemotherapy: the concept of additivity. Int J Radiat Oncol Biol Phys 5: 85-91, 1979), and modified by Kano et al (Kano Y, Suzuki K, Akutsu M, et al: Effects of CPT-11 in combination with other anti-cancer agents in culture. Int J Cancer 50: 604-610, 1992). The criteria of the isobologram are schematically shown in FIG. 7. Three lines (mode I, mode IIa and mode IIb) are IC50 values estimated from growth suppression curves of drug A alone and drug B alone. When the actual IC50 values in combination (data points) are in the area surrounded by these lines (envelope of additivity), that combination is evaluated as additive (point Pb). When the data points fell within the area of left side (point Pa), right side of the envelope (point Pc), and outside the square, the interaction between two drugs was considered to be synergistic, antagonistic, and protective, respectively. Results of E7070 and platinum are as shown in FIG. 8, 9 and 10.

As is evident from FIG. 8, 9 and 10, combined use of E7070 and cisplatin demonstrated synergistic effect only in E7070 pre-treatment treatment, but not in other treatment schedules. On the other hand, the combinations of E7070 with carboplatin or oxaliplatin show synergistic effect in simultaneous treatment. Furthermore, combined use of E7070 oxaliplatin permits their respective effects to be synergistically increased in oxaliplatin pre-treatment schedule. These data indicate that 1) the combination of E7070 with platinum is useful regimen in the aspect of enhancement of antitumor effect synergistically. 2) in the enhancement of antitumor effect, the combination of E7070 with carboplatin or oxaliplatin may have flexibility regarding treatment schedules, compared with that with cisplatin. That is, in the combination of E7070 with carboplatin may be admitted the simultaneous administration of both drugs, and E7070 pre-treatment. E7070 and oxaliplatin may be administered simultaneously, or one of the two may be administered after a predetermined time, and E7070 and oxaliplatin, whichever is administered first, exhibits a synergistic effect.

TABLE 1
Antitumor effect of single or combined drug of E7070 and
CPT-11 in HCT15 xenograft model

| | | Tₓ₄ (days)^{a)} | | mRTV^{b)} | | | Toxicity |
|---|---|---|---|---|---|---|---|
| | | | Two way | | Two way | mRBW^{c)} | Dead/ |
| Treatment | | mean ± S.D. | ANOVA | mean ± S.D. | ANOVA | mean | Treated |
| Control | | 9.5 ± 1.8 | — | 4.41 ± 1.02 (10)^{f} | — | 0.97 (7, 25) | 0/6 |
| E7070^{d)} | 30 mg/kg/day | 18.7 ± 2.0 | — | — | — | 0.92 (7) | 0/6 |
| CPT-11^{e)} | 75 mg/kg/day | 17.8 ± 1.0 | — | — | — | 0.94 (7, 10) | 0/6 |
| E7070 | 30 mg/kg/day | 31.7 ± 4.2 | P < 0.05 | 0.83 ± 0.17 (10) | P < 0.05 | 0.85 (7) | 0/6 |
| +CPT-11 | 75 mg/kg/day | | Synergistic | | Synergistic | | |

^{a)}Tₓ₄: The time in days for tumor size to attain 4 folds of initial tumor size.
^{b)}m (minimum)RTV: RTV = tumor volume on n days after first treatment/tumor volume at the start of treatment, The parentheses indicated the day when RTV was minimal. In control group, RTV on day 10 is indicated.
^{c)}m (minimum)RBW: RBW = body weight on n days after first treatment/body weight at the start of treatment The parentheses indicated the day when RBW was minimal.
^{d)}QDx5 daily for 5 days administration (day 1, 2, 3, 4, 5)
^{e)}Q4Dx3 every 4 days, 3 times administration (day 1, 5, 9)
^{f)}RTV of control group on day 10

TABLE 2
Antitumor effect of single or combined drug of E7070 and
MMC in HCT15 xenograft model

| | | Tₓ₄ (days)^{a)} | | mRTV^{b)} | | | Toxicity |
|---|---|---|---|---|---|---|---|
| | | | Two way | | Two way | mRBW^{c)} | Dead/ |
| Treatment | | mean ± S.D. | ANOVA | mean ± S.D. | ANOVA | mean | Treated |
| Control | | 8.7 ± 1.9 | — | — | — | 0.99 (11) | 0/6 |
| E7070^{d)} | 25 mg/kg/day | 12.7 ± 2.2 | — | — | — | 0.93 (11) | 0/6 |
| MMC^{e)} | 4.19 mg/kg/day | 16.2 ± 3.4 | — | — | — | 0.99 (11) | 0/6 |
| E7070 | 30 mg/kg/day | 26.5 ± 5.3 | P < 0.05 | — | — | 0.93 (11) | 0/6 |
| +MMC | 4.19 mg/kg/day | | Synergistic | | | | |

^{a)}Tₓ₄: The time in days for tumor size to attain 4 folds of initial tumor size.
^{b)}m (minimum)RTV: RTV = tumor volume on n days after first treatment/tumor volume at the start of treatment, In all dose described, tumor regression was not observed.
^{c)}m (minimum)RBW: RBW = body weight on n days after first treatment/body weight at the start of treatment The parentheses indicated the day when RBW was minimal.
^{d)}QDx5 daily for 5 days administration (day 1, 2, 3, 4, 5)
^{e)}QDx1 single dose (day 1)

TABLE 3
Antitumor effect of single or combined drug of E7070 and CPT-11
in SW620 xenograft model

| | | Tₓ₄ (days)^{a)} | | mRTV^{b)} | | | Toxicity |
|---|---|---|---|---|---|---|---|
| | | | Two way | | Two way | mRBW^{c)} | Dead/ |
| Treatment | | mean ± S.D. | ANOVA | mean ± S.D. | ANOVA | mean | Treated |
| Control | | 9.0 ± 2.2 | — | 6.28 ± 4.62 (21) | — | 0.90 (18) | 0/6 |
| E7070^{d)} | 25 mg/kg/day | 30.2 ± 6.1 | — | 0.76 ± 0.42 (12) | — | 0.98 (8) | 0/6 |
| CPT-11^{e)} | 62.5 mg/kg/day | 48.7 ± 29.7 | — | — | — | 0.99 (8) | 0/6 |
| E7070 | 25 mg/kg/day^{d)} | 60.0 ± 8.4 | P > 0.05 | 0.15 ± 0.02 (21) | P < 0.05 | 0.86 (8) | 0/6 |
| +CPT-11 | 62.5 mg/kg/day^{e)} | | additive | | Synergistic | | |
| E7070 | 25 mg/kg/day^{d)} | 65.2 ± 8.5 | P > 0.05 | 0.17 ± 0.09 (21) | P < 0.05 | 0.90 (8) | 0/6 |
| → CPT-11 | 62.5 mg/kg/day^{f)} | | additive | | Synergistic | | |
| CPT-11 | 62.5 mg/kg/day^{e)} | 62.2 ± 10.5 | P > 0.05 | 0.26 ± 0.08 (21) | P < 0.05 | 0.95 (15) | 0/6 |
| → E7070 | 25 mg/kg/day^{g)} | | additive | | Synergistic | | |

^{a)}Tₓ₄: The time in days for tumor size to attain 4 folds of initial tumor size.
^{b)}m (minimum)RTV: RTV = tumor volume on n days after first treatment/tumor volume at the start of treatment, The parentheses indicated the day when RTV was minimal. In control group, RTV on day 21 is indicated.
^{c)}m (minimum)RBW: RBW = body weight on n days after first treatment/body weight at the start of treatment The parentheses indicated the day when RBW was minimal.
^{d)}QDx5 daily for 5 days administration (day 1, 2, 3, 4, 5)
^{e)}Q4Dx3 every 4 days, 3 times administration (day 1, 5, 9)
^{f)}Q4Dx3 every 4 days, 3 times administration (day 6, 10, 14)
^{g)}QDx5 daily for 5 days administration (day 10, 11, 12, 13, 14)

TABLE 4
Antitumor effect of single or combined drug of E7070 and
5-FU in Colo320D. M. xenograft model

| | | Tₓ₄ (days)^{a)} | | mRTV^{b)} | | | Toxicity |
|---|---|---|---|---|---|---|---|
| | | | Two way | | Two way | mRBW^{c)} | Dead/ |
| Treatment | | mean ± S.D. | ANOVA | mean ± S.D. | ANOVA | mean | Treated |
| Control | | 6.0 ± 0.7 | — | — | — | — | 0/6 |
| E7070^{d)} | 30 mg/kg/day | 11.2 ± 1.9 | — | — | — | — | 0/6 |
| 5-FU^{e)} | 60 mg/kg/day | 10.0 ± 0.7 | — | — | — | — | 0/6 |
| E7070 | 30 mg/kg/day^{d)} | 26.0 ± 2.1 | P < 0.05 | — | — | 0.91 (8) | 0/6 |
| +5-FU | 60 mg/kg/day^{e)} | Synergistic | | | | | |
| E7070 | 30 mg/kg/day^{d)} | 15.2 ± 2.6 | p > 0.05 | — | — | 0.99 (8) | 0/6 |
| → 5-FU | 60 mg/kg/day^{f)} | | additive | | | | |
| 5-FU | 60 mg/kg/day^{e)} | 8.6 ± 0.89 | p > 0.05 | — | — | 0.99 (15) | 0/6 |
| → E7070 | 30 mg/kg/day^{g)} | | additive | | | | |

^{a)}Tₓ₄: The time in days for tumor size to attain 4 folds of initial tumor size.
^{b)}m (minimum)RTV: RTV = tumor volume on n days after first treatment/tumor volume at the start of treatment, “—”: Tumor regression was not observed.
^{c)}m (minimum)RBW: RBW = body weight on n days after first treatment/body weight at the start of treatment The parentheses indicated the day when RBW was minimal. “—”: body weight loss was not observed.
^{d)}QDx5 daily for 5 days administration (day 1, 2, 3, 4, 5)
^{e)}Q4Dx3 every 4 days, 3 times administration (day 1, 5, 9)
^{f)}Q4Dx3 every 4 days, 3 times administration (day 6, 10, 14)
^{g)}QDx5 daily for 5 days administration (day 10, 11, 12, 13, 14)

TABLE 5
Antitumor effect of single or combined drug of E7070 and
CDDP in LU-99 xenograft model

| | | Tₓ₄ (days)^{a)} | | mRTV^{b)} | | | Toxicity |
|---|---|---|---|---|---|---|---|
| | | | Two way | | Two way | mRBW^{c)} | Dead/ |
| Treatment | | mean ± S.D. | ANOVA | mean ± S.D. | ANOVA | mean | Treated |
| Control | | 8.3 ± 1.2 | — | — | — | — | 0/6 |
| E7070^{d)} | 25 mg/kg/day | 18.2 ± 2.3 | — | 0.75 ± 0.16 | — | — | 0/6 |
| CDDP^{e)} | 7.5 mg/kg/day | 10.7 ± 0.5 | — | — | — | 0.95 (5) | 0/6 |
| E7070 | 25 mg/kg/day^{d)} | 26.0 ± 5.1 | P < 0.05 | 0.97 ± 0.21 | P > 0.05 | 0.82 (8) | 0/6 |
| +CDDP | 7.5 mg/kg/day^{e)} | | Synergistic | | Additive | | |
| E7070 | 25 mg/kg/day^{d)} | 28.0 ± 8.2 | P < 0.05 | 0.82 ± 0.22 | P > 0.05 | 0.86 (8) | 0/6 |
| → CDDP | 7.5 mg/kg/day^{f)} | | Synergistic | | Additive | | |
| CDDP | 7.5 mg/kg/day^{e)} | 20.3 ± 2.2 | P > 0.05 | 0.99 ± 0.21 | P > 0.05 | 0.88 (8) | 0/6 |
| → E7070 | 25 mg/kg/day^{g)} | | Additive | | Additive | | |

^{a)}T_{x4:} The time in days for tumor size to attain 4 folds of initial tumor size.
^{b)}m (minimum)RTV: RTV = tumor volume on n days after first treatment/tumor volume at the start of treatment, “—”: Tumor regression was not observed.
^{c)}m (minimum)RBW: RBW = body weight on n days after first treatment/body weight at the start of treatment The parentheses indicated the day when RBW was minimal.
^{d)}QDx5 daily for 5 days administration (day 1, 2, 3, 4, 5)
^{e)}QDx1 single dose (day 1)
^{f)}QDx1 single dose (day 6)
^{g)}QDx5 daily for 5 days administration (day 2, 3, 4, 5, 6)

TABLE 6
Antitumor effect of single or combined drug of E7070 and
capecitabine in HBC4 xenograft model

| Treatment | | | Antitumor effect | | | | | | |
|---|---|---|---|---|---|---|---|---|---|
| | E7070 | Capecitabine | Tx4 | T-C | 2 Way | | 2 Way | | |
| | (mg/kg/day) | (mmol/kg/day) | (days) | (days) | ANOVA | mRTV | ANOVA | PR | CR |
| Control | — | — | 22.0 ± 3.8 | — | — | — | — | 0/5 | 0/5 |
| E7070 alone | 30.625 (87.5% (MTD) | — | 50.0 ± 8.1 | 28.0 | — | 0.24 ± 0.07 | — | 6/6 | 0/6 |
| Capecitabine | — | 1.3125 (87.5% MTD) | 29.0 ± 2.5 | 7.0 | — | — | — | 0/6 | 0/6 |
| alone | | | | | | | | | |
| E7070 + | 30.625 | 1.3125 | 51.2 ± 16.4 | 29.2 | P = 0.492 | 0.18 ± 0.07 | P = 0.009 | 6/6 | 1/6 |
| Capecitabine | | | | | | | | | |

m (minimum)RTV: RTV = tumor volume on n days after first treatment/tumor volume at the start of treatment.
“—” means no tumor regression.
m (minimum)RBW: RBW = body weight on n days after first treatment/body weight at the start of treatment
Partial response (PR): The incidence of tumor regression (more than 50% of tumor regression).
Complete response (CR): The incidence of tumor free mice
RBW < 0.80: The incidence of more than 20% body weight loss (undue toxicity)

1. A medicinal composition comprising a sulfonamide compound, a sulfonate compound or a salt of them (sulfone compound (I)), which is represented by the following formula: 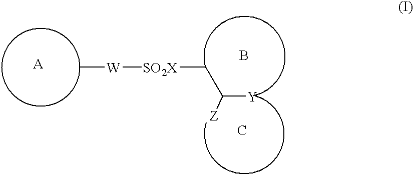
(wherein ring A represents a monocyclic aromatic ring or a bicyclic aromatic ring, each of which may have a substituent group; ring B represents a 6-membered unsaturated hydrocarbon ring or a 6-membered unsaturated hetero-ring containing one nitrogen atom, each of which may have a substituent group; ring C represents a 5-membered hetero-ring containing one or two nitrogen atoms, and the ring C may have a substituent group; W represents a single bond or —CH═CH—; X represents —N(R¹)—(wherein R¹ represents a hydrogen atom or a lower alkyl group) or an oxygen atom; Y represents a carbon atom or a nitrogen atom; Z represents —N(R²)— (R² represents a hydrogen atom or a lower alkyl group) or a nitrogen atom, provided that the above-defined compounds exclude compounds in the following cases (a) and (b):
(a) ring A is 4-methylbenzene; W is a single bond; X is —NH—; ring B is methoxybenzene; and ring C is unsubstituted imidazole; and (b) ring A is 4-(acetamide) benzene or 4-aminobenzene; W is a single bond; X is —NH—; ring B is unsubstituted benzene; and ring C is unsubstituted pyrazole), combined with and at least one substance (III) selected from (1) irinotecan hydrochloride trihydrate; (2) mitomycin C; (3) 5-fluorouracil; (4) cisplatin; (5) gemcitabine hydrochloride; (6) doxorubicin; (7) taxol; and (8) a salt of the above-mentioned (1) to (7).

2. The medicinal composition according to claim 1, wherein in the above formula (I), ring A is benzene or pyridine, each of which may have a substituent group; ring B is benzene which may have a substituent group, ring C is pyrrole which may have a substituent group; W is a single bond; and each of X and Z is —NH—.

3. The medicinal composition according to claim 1, which is an anticancer agent.

4. The medicinal composition according to claim 3, which comprises N-(3-chloro-1H-indol-7-yl)-4-sulfamoylbenzenesulfonamide or a salt thereof, and at least one substance selected from (1) irinotecan hydrochloride trihydrate; (2) mitomycin C; (3) 5-fluorouracil; (4) cisplatin; (5) gemcitabine hydrochloride; (6) doxorubicin; (7) taxol; and (8) a salt of the above-mentioned (1) to (7).

5. The medicinal composition according to claim 3, which comprises N-(3-chloro-1H-indol-7-yl)-4-sulfamoylbenzenesulfonamide or a salt thereof, and irinotecan hydrochloride trihydrate or a salt thereof.

6. The medicinal composition according to claim 3, which comprises N-(3-chloro-1H-indol-7-yl)-4-sulfamoylbenzenesulfonamide or a salt thereof, and mitomycin C or a salt thereof.

7. The medicinal composition according to claim 3, which comprises N-(3-chloro-1H-indol-7-yl)-4-sulfamoylbenzenesulfonamide or a salt thereof, and 5-fluorouracil or a salt thereof.

8. The medicinal composition according to claim 3, which comprises N-(3-chloro-1H-indol-7-yl)-4-sulfamoylbenzene sulfonamide or a salt thereof, and cisplatin or a salt thereof.

9. The medicinal composition according to claim 3, which comprises N-(3-chloro-1H-indol-7-yl)-4-sulfamoylbenzenesulfonamide or a salt thereof, and gemcitabine hydrochloride or a salt thereof.

10. The medicinal composition according to claim 3, which comprises N-(3-chloro-1H-indol-7-yl)-4-sulfamoylbenzene-sulfonamide or a salt thereof, and doxorubicin or a salt thereof.

11. The medicinal composition according to claim 3, which comprises N-(3-chloro-1H-indol-7-yl)-4-sulfamoylbenzene-sulfonamide or a salt thereof, and taxol or a salt thereof.

12. The medicinal composition according to claim 3, which comprises N-(3-chloro-1H-indol-7-yl)-4-sulfamoylbenzene-sulfonamide or a salt thereof, irinotecan hydrochloride trihydrate or a salt thereof, and cisplatin or a salt thereof.

13. The medicinal composition according to claim 3, which comprises N-(3-chloro-1H-indol-7-yl)-4-sulfamoylbenzene-sulfonamide or a salt thereof, irinotecan hydrochloride trihydrate or a salt thereof, 5-fluorouracil or a salt thereof, and cisplatin or a salt thereof.

14. Use of N-(3-chloro-1H-indol-7-yl)-4-sulfamoylbenzenesulfonamide or a salt thereof for producing a medicinal composition comprising it in combination with at least one substance selected from (1) irinotecan hydrochloride trihydrate; (2) mitomycin C; (3) 5-fluorouracil; (4) cisplatin; (5) gemcitabine hydrochloride; (6) doxorubicin; (7) taxol; and (8) a salt of the above-mentioned (1) to (7).

15. Use of at least one substance selected from (1) irinotecan hydrochloride trihydrate; (2) mitomycin C; (3) 5-fluorouracil; (4) cisplatin; (5) gemcitabine hydrochloride; (6) doxorubicin; (7) taxol; and (8) a salt of the above-mentioned (1) to (7), for producing a medicinal composition comprising it in combination with N-(3-chloro-1H-indol-7-yl)-4-sulfamoylbenzenesulfonamide or a salt thereof.

16. Use of N-(3-chloro-1H-indol-7-yl)-4-sulfamoylbenzenesulfonamide or a salt thereof, and at least one substance selected from (1) irinotecan hydrochloride trihydrate; (2) mitomycin C; (3) 5-fluorouracil; (4) cisplatin; (5) gemcitabine hydrochloride; (6) doxorubicin; (7) taxol; and (8) a salt of the above-mentioned (1) to (7), for producing an agent for preventing or treating a cancer.

17. A method for preventing or treating a cancer, by administrating an effective amount of the medicinal composition of claim 3 to a patient.

18. A method for preventing or treating a cancer, by administering N-(3-chloro-1H-indol-7-yl)-4-sulfamoylbenzenesulfonamide or a salt thereof to a patient, and then administering, after a predetermined time, at least one substance selected from (1) irinotecan hydrochloride trihydrate; (2) mitomycinC; (3) 5-fluorouracil; (4) cisplatin; (5) gemcitabine hydrochloride; (6) doxorubicin; (7) taxol; and (8) a salt of the above-mentioned (1) to (7) to the patient.

19. A method for preventing or treating a cancer, by administering at least one substance selected from (1) irinotecan hydrochloride trihydrate; (2) mitomycin C; (3) 5-fluorouracil; (4) cisplatin; (5) gemcitabine hydrochloride; (6) doxorubicin; (7) taxol; and (8) a salt of the above-mentioned (1) to (7) to a patient, and then administering, after a predetermined time, N-(3-chloro-1H-indol-7-yl)-4-sulfamoylbenzenesulfonamide or a salt thereof to the patient.

20. A pharmaceutical kit for administering effective amounts of the sulfone compound (I) of claim 1 and the substance (III) of claim 1 simultaneously or separately to a patient.

21. A pharmaceutical product comprising the sulfone compound (I) of claim 1 combined with the substance (III) of claim 1.

22. A combination comprising the sulfone compound (I) of claim 1 combined with the substance (III) of claim 1.

23. A method for preventing or treating a cancer, by administering effective amounts of the sulfone compound (I) of claim 1 combined with the substance (III) of claim 1 simultaneously or separately to a patient.

24. A medicinal composition comprising effective amounts of the sulfone compound (I) of claim 1 and the substance (III) of claim 1.

25. A medicinal composition comprising a sulfonamide compound, a sulfonate compound or a salt of them (sulfone compound (I)), which is represented by the following formula: 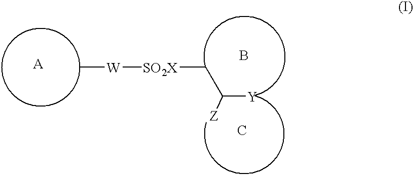
(wherein ring A represents a monocyclic aromatic ring or a bicyclic aromatic ring, each of which may have a substituent group; ring B represents a 6-membered unsaturated hydrocarbon ring or a 6-membered unsaturated hetero-ring containing one nitrogen atom, each of which may have a substituent group; ring C represents a 5-membered hetero-ring containing one or two nitrogen atoms, and the ring C may have a substituent group; W represents a single bond or —CH═CH—; X represents —N(R¹)—(wherein R¹ represents a hydrogen atom or a lower alkyl group) or an oxygen atom; Y represents a carbon atom or a nitrogen atom; Z represents —N(R²)—(R² represents a hydrogen atom or a lower alkyl group) or a nitrogen atom, provided that the above-defined compounds exclude compounds in the following cases (a) and (b):
(a) ring A is 4-methylbenzene; W is a single bond; X is —NH—; ring B is methoxybenzene; and ring C is unsubstituted imidazole; and (b) ring A is 4-(acetamide)benzene or 4-aminobenzene; W is a single bond; X is —NH—; ring B is unsubstituted benzene; and ring C is unsubstituted pyrazole), combined with and at least one substance (IV) selected from (1) irinotecan hydrochloride trihydrate; (2) mitomycin C; (3) 5-fluorouracil; (4) cisplatin; (5) gemcitabine hydrochloride; (6) doxorubicin; (7) taxol; (8) carboplatin; (9) oxaliplatin; (10) capecitabine; and (11) a salt of the above-mentioned (1) to (10).

26. The medicinal composition according to claim 25, wherein in the above formula (I), ring A is benzene or pyridine, each of which may have a substituent group; ring B is benzene which may have a substituent group, ring C is pyrrole which may have a substituent group; W is a single bond; and each of X and Z is —NH—.

27. The medicinal composition according to claim 25, which is an anticancer agent.

28. The medicinal composition according to claim 27, which comprises N-(3-chloro-1H-indol-7-yl)-4-sulfamoylbenzenesulfonamide or a salt thereof, and at least one substance selected from (1) irinotecan hydrochloride trihydrate; (2) mitomycin C; (3) 5-fluorouracil; (4) cisplatin; (5) gemcitabine hydrochloride; (6) doxorubicin; (7) taxol; (8) carboplatin; (9) oxaliplatin; (10) capecitabine; and (11) a salt of the above-mentioned (1) to (10).

29. The medicinal composition according to claim 27, which comprises N-(3-chloro-1H-indol-7-yl)-4-sulfamoylbenzenesulfonamide or a salt thereof, and carboplatin or a salt thereof.

30. The medicinal composition according to claim 27, which comprises N-(3-chloro-1H-indol-7-yl)-4-sulfamoylbenzenesulfonamide or a salt thereof, and oxaliplatin or a salt thereof.

31. The medicinal composition according to claim 27, which comprises N-(3-chloro-1H-indol-7-yl)-4-sulfamoylbenzenesulfonamide or a salt thereof, and capecitabine or a salt thereof.

32. The medicinal composition according to claim 27, which comprises N-(3-chloro-1H-indol-7-yl)-4-sulfamoylbenzenesulfonamide or a salt thereof, 5-fluorouracil or a salt thereof, and oxaliplatin or a salt thereof.

33. Use of N-(3-chloro-1H-indol-7-yl)-4-sulfamoylbenzenesulfonamide or a salt thereof for producing a medicinal composition comprising it in combination with at least one substance selected from (1) irinotecan hydrochloride trihydrate; (2) mitomycin C; (3) 5-fluorouracil; (4) cisplatin; (5) gemcitabine hydrochloride; (6) doxorubicin; (7) taxol; (8) carboplatin; (9) oxaliplatin; (10) capecitabine; and (11) a salt of the above-mentioned (1) to (10).

34. Use of at least one substance selected from (1) irinotecan hydrochloride trihydrate; (2) mitomycin C; (3) 5-fluorouracil; (4) cisplatin; (5) gemcitabine hydrochloride; (6) doxorubicin; (7) taxol; (8) carboplatin; (9) oxaliplatin; (10) capecitabine; and (11) a salt of the above-mentioned (1) to (10), for producing a medicinal composition comprising it in combination with N-(3-chloro-1H-indol-7-yl)-4-sulfamoylbenzenesulfonamide or a salt thereof.

35. Use of N-(3-chloro-1H-indol-7-yl)-4-sulfamoylbenzenesulfonamide or a salt thereof, and at least one substance selected from (1) irinotecan hydrochloride trihydrate; (2) mitomycin C; (3) 5-fluorouracil; (4) cisplatin; (5) gemcitabine hydrochloride; (6) doxorubicin; (7) taxol; (8) carboplatin; (9) oxaliplatin; (10) capecitabine; and (11) a salt of the above-mentioned (1) to (10), for producing an agent for preventing or treating a cancer.

36. A method for preventing or treating a cancer, by administrating an effective amount of the medicinal composition of claim 27 to a patient.

37. A method for preventing or treating a cancer, by administering N-(3-chloro-1H-indol-7-yl)-4-sulfamoylbenzenesulfonamide or a salt thereof to a patient, and then administering, after a predetermined time, at least one substance selected from (1) irinotecan hydrochloride trihydrate; (2) mitomycin C; (3) 5-fluorouracil; (4) cisplatin; (5) gemcitabine hydrochloride; (6) doxorubicin; (7) taxol; (8) carboplatin; (9) oxaliplatin; (10) capecitabine; and (11) a salt of the above-mentioned (1) to (10) to the patient.

38. A method for preventing or treating a cancer, by administering at least one substance selected from (1) irinotecan hydrochloride trihydrate; (2) mitomycin C; (3) 5-fluorouracil; (4) cisplatin; (5) gemcitabine hydrochloride; (6) doxorubicin; (7) taxol; (8) carboplatin; (9) oxaliplatin; (10) capecitabine; and (11) a salt of the above-mentioned (1) to (10) to a patient, and then administering, after a predetermined time, N-(3-chloro-1H-indol-7-yl)-4-sulfamoylbenzenesulfonamide or a salt thereof to the patient.

39. A pharmaceutical kit for administering effective amounts of the sulfone compound (I) of claim 25 and the substance (IV) of claim 25 simultaneously or separately to a patient.

40. A pharmaceutical product comprising the sulfone compound (I) of claim 25 combined with the substance (IV) of claim 25.

41. A combination comprising the sulfone compound (I) of claim 25 combined with the substance (IV) of claim 25.

42. A method for preventing or treating a cancer, by administering effective amounts of the sulfone compound (I) of claim 25 combined with the substance (IV) of claim 25 simultaneously or separately to a patient.

43. A medicinal composition comprising effective amounts of the sulfone compound (I) of claim 25 and the substance (IV) of claim 25.
